(19) United States
(12) Patent Application Publication
(10) Pub. No.: US 2007/0010543 A1
(43) Pub. Date: Jan. 11, 2007

(54) Compositions and methods for treating gastrointestinal hypomotility and associated disorders

(75) Inventor: Ted T. Ashburn, Boston, MA (US)

Correspondence Address: LAHIVE & COCKFIELD, LLP, ONE POST OFFICE SQUARE, BOSTON, MA, 02109-2127, US

(73) Assignee: Dynogen Pharmaceuticals, Inc., Waltham, MA (US)

(21) Appl. No.: 11/479,139

(22) Filed: Jun. 30, 2006

Related U.S. Application Data
(60) Provisional application No. 60/696,098, filed on Jul. 1, 2005.

(51) Int. Cl. A61K 31/485; A61K 31/4743

(52) U.S. Cl. 514/282; 514/301

(57) ABSTRACT

This invention relates to methods for treating, preventing and/or managing GI hypomotility or a disorder associated with GI hypomotility in a subject including administering to the subject two compounds, where the first compound is a 5HT₃ agonist and where the combination of the two compounds results in an increase in GI motility. Also provided are compositions and kits for use in methods of the invention.

### PRIORITY

This application claims the benefit of priority to U.S. Provisional Patent Application No. 60/696,098, filed Jul. 1, 2005, the entire content of which is incorporated by reference herein.

### BACKGROUND

Gastrointestinal (GI) motility regulates the orderly movement of ingested material through the gut to ensure adequate absorption of nutrients, electrolytes and fluids and expulsion of waste materials. Normal transit through the esophagus, stomach, small intestine and colon depends, at least in part, on the coordinated, regional control of intraluminal pressure and several sphincters that regulate forward movement and prevent back-flow of GI contents. The normal GI motility pattern can be impaired by a variety of circumstances including disease, surgery, certain medications and old age.

Various disorders associated with GI hypomotility, including, for example, gastroesophageal reflux disease (GERD), nocturnal gastroesophageal reflux disease (n-GERD); dyspepsia, constipation including slow transit constipation, normal transit constipation, acute constipation, chronic idiopathic constipation, constipation associated with irritable bowel syndrome and constipation due to increased tone of the large intestine, ileus and post-operative ileus, narcotic bowel syndrome, gastroparesis including diabetic gastroparesis and intestinal pseudo-obstruction gastroparesis, Hirschsprung's disease, decreased peristalsis of the esophagus and/or stomach and/or the small and/or the large intestine, esophagitis, non-ulcer dyspepsia, pseudo-obstruction of the bowels and/or the colon, impaired colonic transit, epigastric pain, recurrent nausea and vomiting, anorexia nervosa, dyskinesias of the biliary system, tachygastria and problems of gastric emptying due to tachygastria, intestinal spasms and cramps, irritable bowel syndrome with diarrhea, peptic ulcer diseases, inflammatory diseases of the stomach and bowel including gastritis, inflammatory bowel disease, duodenitis, intestinal and post-operative intestinal atony, and drug-induced delayed transit.

Primary treatment of GI hypomotility includes, for example, dietary manipulation and administration of antiemetic and prokinetic agents. However, none of these treatments are fully safe and/or effective. Prokinetic drugs act to stimulate GI motility, for example, by direct action on smooth muscle or by an action on the myenteric plexus. However, there are currently no prokinetic drugs available which are both effective and safe and with minimal side effects. For example, the prokinetic drug, ZELNORM® (tegaserod), the only drug approved for constipation in the United States, is only minimally efficacious and has been reported to result in serious consequences of diarrhea, hypovolemia, hypotension, orthostatic intolerance and syncope in some patients. Ischemic colitis and other forms of intestinal ischemia have also been reported in patients receiving ZELNORM®, and in some cases, hospitalization was required. In addition, the prokinetic agent PROPULSID® (cisapride) has been used widely off label for many of the above mentioned disorders associated with GI hypomotility. However, this drug was withdrawn from the market in the year 2000 for causing serious cardiac arrhythmias including ventricular tachycardia, ventricular fibrillation, torsades de pointes, and QT prolongation. Further, the use of the dopamine antagonists, metoclopramide and domperidone, as prokinetic agents, is associated with poor tolerability due to undesirable CNS and cardiovascular effects, including tardive dyskinesia and QT prolongation.

Therefore, there is a need for the development of novel therapies that are more effective and safe for the prophylactic or curative treatment of GI hypomotility and associated disorders.

### SUMMARY

The present invention relates to methods for treating, preventing and/or managing GI hypomotility, and disorders associated therewith, in a subject including administering to the subject at least two compounds, where the combination of the at least two compounds results in an increase in GI motility, for example, by promoting (i.e., increasing, enhancing or inducing) physical propulsions anywhere in the GI tract and/or increasing GI secretions, for example, increasing the rate and/or amounts of secretions anywhere in the GI tract. Because of the resultant increase in GI motility by compositions of the invention, lower amounts of each compound are used in the compositions for treating, preventing and/or managing GI hypomotility, thereby avoiding undesirable or harmful side effects associated with use of higher amounts of these compounds that are normally used when each compound is used alone. Furthermore, in those subjects who are either unresponsive or minimally responsive to each compound when used alone at suggested doses and/or those who experience side effects at suggested doses, normal or lower amounts of each compound when given in combination could provide for greater efficacy and/or tolerability.

Two or more compounds described herein can be constituted in a single composition, such that administration of the composition results in an increase in GI motility, for example, either by promoting physical propulsions anywhere in the GI tract and/or increasing secretions anywhere in the GI tract, or the two or more compounds may be administered separately. Compounds may either be co-administered (i.e., at the same time) or be administered sequentially (i.e., one after the other). The resultant increase in GI motility can either be synergistic or additive. In some embodiments, the resultant increase in GI motility is synergistic. This disclosure provides compositions for treating various disorders discussed herein. Examples of various disorders that can be treated with the compositions of the present invention include, for example, gastroesophageal reflux disease (GERD), dyspepsia, constipation including slow transit constipation, normal transit constipation, acute constipation, chronic idiopathic constipation, opioid-induced constipation, constipation associated with irritable bowel syndrome and constipation due to increased tone of the large intestine, ileus and post-operative ileus, narcotic bowel syndrome, gastroparesis including diabetic gastroparesis and intestinal pseudo-obstruction gastroparesis, Hirschsprung's disease, decreased peristalsis of the esophagus and/or stomach and/or the small and/or the large intestine, esophagitis, non-ulcer dyspepsia, pseudo-obstruction of the bowels and/or the colon, impaired colonic transit, epigastric pain, recurrent nausea and vomiting, anorexia nervosa, dyskinesias of the biliary system, tachygastria and problems of gastric emptying due to tachygastria, intestinal spasms and cramps, irritable bowel syndrome with diarrhea, peptic ulcer diseases, inflammatory diseases of the stomach and bowel including gastritis, duodenitis, inflammatory bowel disease, intestinal and post-operative intestinal atony, and drug-induced delayed transit.

In some embodiments, compositions described herein include a first compound having a 5-HT₃ receptor agonist activity or a pharmaceutically acceptable salt, hydrate or solvate thereof, and at least one second compound, where the combination of the first compound and the at least one second compound results in an increase in GI motility, for example, either by promoting physical propulsions anywhere in the GI tract and/or increasing secretions anywhere in the GI tract. An added benefit of such compositions is that they would alleviate visceral pain and bloating associated with GI hypomotility disorders such as irritable bowel syndrome (IBS).

Examples of a compound having 5-HT₃ receptor agonist activity include, but are not limited to, MKC-733, also referred to as Dynogen Development Program 733 (DDP733) or pumosetrag, (i.e., the monohydrochloride salt of (R)-N-1-azabicyclo[2.2.2]oct-3-yl-4,7-dihydro-7-oxothieno[3,2-b]pyridine-6-carboxamide), thiazole derivatives, thieno[3,2-b]pyridine derivatives, YM 31636, and equivalents thereof.

In some embodiments, the at least one second compound is a compound chosen from: compounds having 5-HT₄ receptor agonist activity (e.g., tegaserod, cisapride, prucalopride, SL 65.0155, ATI-7505, and TD-2749); compounds having both 5-HT₄ receptor agonist activity and 5-HT₃ receptor antagonist activity (e.g., mosapride, renzapride, and E-3620); compounds having both 5-HT₄ receptor agonist activity and dopamine receptor antagonist activity (e.g., metoclopramide); compounds having both 5-HT₄ receptor agonist activity and D₂-receptor antagonist activity (e.g., itopride); compounds having D₂-receptor antagonist activity (e.g., chlorpromazine, prochlorperazine, haloperidol, and alizapride); compounds having motilin receptor agonist activity (e.g., erythromycin, mitemcinal, and atilmotin); compounds having GABA-B receptor agonist activity (e.g., baclofen and XP-19986); compounds having corticotropin-releasing factor (CRF-1) receptor antagonist activity (e.g., CP-316311, TS-041, 876008, ONO-2333MS, MG-561 and NBI-34041); compounds having opioid receptor antagonist activity (e.g., naltrexone, PTI-901, alvimopan, methylnaltrexone, and naloxone); compounds having alpha 2 adrenoreceptor agonist activity (e.g., clonidine, tizanidine, and guanfacine); compounds having tachykinin (NK₁, NK₂, and/or NK₃) receptor antagonist activity (e.g., nepadutant, saredutant, talnetant, and osanetant); compounds having muscarinic cholinergic receptor agonist activity (e.g. bethanechol and carbachol) and acetylcholinesterase inhibitors (e.g., neostigmine); compounds having mixed serotonin and noradrenaline reuptake inhibitor activity (e.g., milnacipran, venlafaxine, desvenlafaxine, sibutramine and duloxetine); benzodiazepine-like molecules (e.g., dextofisopam, levotofisopam, diazepam, lorazepam, alprazolam and clonazepam); compounds having nitric oxide synthase (NOS) inhibitory activity (e.g., SC-81490, targinine and 274150); cannabinoid receptor modulators (e.g., dronabinol, nabilone, rimonabant, cannabidiol, and SAB-378); compounds having reversible cholecystokinin 1 (CCK1) receptor antagonist activity (e.g., dexloxiglumide, and devazepide); compounds that activate chloride channels (e.g. lubiprostone); compounds having guanylate cyclase receptor agonist activity (e.g., MD-1100); glucagon-like peptide (GLP) analogs (e.g. GTP-010). Compositions described herein typically increase GI motility including esophageal and colonic motility, and peristaltic wave amplitude which result in, for example, an increase in physical propulsions in the GI tract.

In some embodiments, the at least one second compound is an opioid receptor antagonist, including, but not limited to, for example, antagonist of a μ-type opioid receptor, antagonist of a δ-type opioid receptor, and antagonist of a κ-type opioid receptor.

In some embodiments, an opioid receptor antagonist is an antagonist of a μ-type opioid receptor, such as, for example, naltrexone and alvimopan (also knows as Entereg and ADL 8-2698).

In some embodiments, a composition featured herein comprises therapeutically effective doses of a compound having a 5-HT₃ receptor agonist activity (e.g., DDP733) and a compound having a opioid receptor antagonist activity (e.g., naltrexone or alvimopan). In a particular embodiment, a composition featured herein comprises therapeutically effective doses of DDP733 and naltrexone. In another embodiment, a composition featured herein comprises therapeutically effective doses of DDP733 and alvimopan (also knows as Entereg and ADL 8-2698).

### BRIEF DESCRIPTION OF THE DRAWINGS

FIG. 1 is a bar graph depicting the results from an exemplary experiment demonstrating that a compound having 5-HT₃ receptor agonist activity (i.e., DDP733) in combination with a compound having opioid antagonist activity (i.e, naltrexone) leads to the reversal of a morphine-induced decrease in pellet propulsion in a guinea pig ex vivo colon preparation. Y-axis represents motility, measured as a percentage of saline control. These data demonstrate that the motility in the presence of morphine+naltrexone+DDP733 was significantly higher relative to morphine alone.

FIG. 2 is a bar graph depicting the results based on an addivity test demonstrating that DDP733 and naltrexone have an effect on motility similar to what would be predicted theoretically.

### DETAILED DESCRIPTION

The present invention features methods of treating, preventing and/or managing GI hypomotility and disorders associated therewith, and compositions for use in such methods. In particular, methods described herein are directed to treatment, prevention and/or management of disorders associated with GI hypomotility, for example, disorders where GI motility is decreased abnormally. It is understood that compositions and methods described herein may either be used to treat, prevent or manage a disorder associated with GI hypomotility or for treating and/or preventing GI hypomotility itself. In some instances, GI hypomotility is a component of a disorder. It is understood that abnormal GI motility may either be a cause or a symptom of a disorder. It is further understood that GI hypomotility may be associated with visceral pain, as in IBS with constipation, and that alleviation of GI hypomotility may confer added benefit for the relief of visceral pain in such conditions.

The neurotransmitter serotonin was first discovered in 1948 and has subsequently been the subject of substantial scientific research. Serotonin, also referred to as 5-hydroxytryptamine (5-HT), acts both centrally and peripherally on all serotonin receptors. Currently, fourteen subtypes of serotonin receptors are recognized and delineated into seven families, designated 5-HT₁ through 5-HT₇. A review of the nomenclature and classification of the 5-HT receptors can be found in, for example, Neuropharmacology 33: 261-273 (1994); and Pharmacol. Rev., 46:157-203 (1994), the entire contents of which are incorporated herein by reference. These subtypes share close sequence homology and display some similarities in their specificity for particular ligands. While these receptors all bind serotonin, they initiate different signaling pathways to perform different functions. For example, in the GI tract, serotonin is known to activate submucosal intrinsic nerves via stimulation of 5-HT_{1P}, 5-HT3 and 5-HT₄ receptors, resulting in, for example, the initiation of peristaltic and secretory reflexes.

5-HT₃ receptors are ligand-gated ion channels that are distributed extensively on enteric neurons in the human GI tract, as well as other peripheral and central tissues.

As used herein, the term “5-HT₃ receptors” refers to naturally occurring 5-HT₃ receptors (e.g., mammalian 5-HT₃ receptors (e.g., human (Homo sapiens) 5-HT₃ receptors, murine (e.g., rat, mouse) 5-HT₃ receptors)) and to proteins having an amino acid sequence which is substantially identical to the sequence of a corresponding naturally occurring 5-HT₃ receptors (e.g., recombinant proteins). In some embodiments, a protein having 5-HT₃ receptor activity has an amino acid sequence at least 60%, or at least 70%, or at least 80%, or at least 90%, or at least 95%, or at least 96%, or at least 97%, or at least 98%, or at least 99% or more identical to a naturally occurring 5-HT₃ receptor. The term “5-HT₃ receptors” includes naturally occurring variants, such as polymorphic or allelic variants and splice variants having 5-HT₃ receptor activity. In addition, the term “5-HT3 receptor,” as used herein, encompasses a truncated, modified, mutated receptor, or any molecule comprising part or all of the sequences of a receptor, or subunits of a receptor, having 5-HT₃ receptor activity.

As used herein, “a compound having 5-HT₃ receptor agonist activity” refers to a substance (e.g., a molecule, a compound) which promotes (induces, enhances or increases) at least one functional consequence of 5-HT₃ receptor activation. In some embodiments, a compound having 5-HT₃ receptor agonist activity binds a 5-HT₃ receptor (i.e., is a 5-HT₃ receptor agonist). In certain embodiments, the agonist is a 5-HT₃ receptor partial agonist. Partial agonist, as used herein, refers to an agonist which is unable to produce maximal activation of a 5-HT₃ receptor as defined using the endogenous ligand, 5-HT, no matter how high a concentration is used. A compound having 5-HT₃ receptor agonist activity (e.g., a 5-HT₃ receptor agonist) can be identified and activity assessed by any suitable method. For example, the binding affinity of a 5-HT₃ receptor agonist to the 5-HT₃ receptor can be determined by the ability of the compound to displace a radiolabeled ligand with high affinity for the 5-HT₃ receptor, such as [³H]granisetron from tissues in which there is a high density of 5-HT₃ receptors, such as rat cerebral cortex membranes (Cappelli et al., J. Med. Chem., 42(9): 1556-1575 (1999)). In addition, the agonist activity of various compounds can be assessed using various in vitro functional assays, for example, by measuring the activation of 5-HT₃ receptors on guinea-pig myenteric neurons using electrophysiological techniques (Zhou & Galligan J., Pharmacol. Exp. Therap., 290: 803-810 (1999)).

In some embodiments, a compound having 5-HT₃ receptor agonist activity is MKC-733, also referred to as DDP733 and pumosetrag, and derivatives thereof, such as described in U.S. Pat. No. 5,352,685 and U.S. Patent Publication No. 20050059704, the entire contents of which are incorporated herein by reference. In other embodiments, a compound having 5-HT₃ receptor agonist activity is a thieno[3,2-b]pyridine derivative such as those described in, U.S. Pat. No. 5,352,685, the entire content of which is incorporated herein by reference. Examples of a compound having 5-HT₃ receptor agonist activity, including MKC-733 and derivatives thereof, are also discussed in PCT publication WO 01/37824, which discusses use of such compounds for relaxation of the fundus, and US publication No. 20030130304, which discusses use of thieno[3,2-b]pyridinecarboxamide derivatives in the treatment and/or prevention of GERD. The entire contents of these publications are incorporated by reference herein. In yet other embodiments, a compound having 5-HT₃ receptor agonist activity is a condensed thiazole derivative such as those described in U.S. Pat. No. 5,565,479, the entire content of which is incorporated herein by reference.

In some embodiments, a compound having 5-HT₃ receptor agonist activity is represented by the formula below (Formula I): 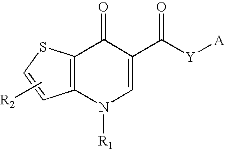
where R₁ represents hydrogen, a C₁-C₆ alkyl group, a C₂-C₆ alkenyl group, a C₂-C₆ alkynyl group, a C₃-C₈ cycloalkyl group, a C₆-C₁₂ aryl group or a C₇-C₁₈ aralkyl group;
R2 represents hydrogen, a C₁-C₆ alkyl group, a halogen, hydroxyl, a C₁-C₆ alkoxy group, an amino group, a C₁-C₆ alkylamino group, a nitro group, a mercapto or a C₁-C₆ alkylthio group;
Y represents —O— or 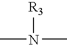
R₃ represents hydrogen or a C₁-C₆ alkyl group; and
A is represented by 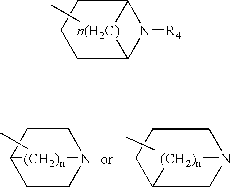
   where n is an integer from 1 to about 4; R₄ represents hydrogen, a C₁-C₆ alkyl group, a C₃-C₈ cycloalkyl group or a C₇-C₁₈ aralkyl group; or a pharmaceutically acceptable salt, solvate, hydrate or N-oxide derivative thereof.

It is understood that when R, of Formula I is hydrogen, compounds having the tautomeric form represented by Formula IA below are also included.

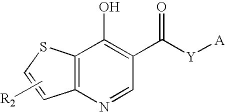

Likewise, it is understood that Formula 1A includes the tautomeric form represented by Formula I when R₁ is hydrogen.

In some embodiments, compounds represented by Formula I can be N-oxide derivatives.

In another embodiment of Formula I, Y represents —O— or 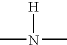
where R₁ represents hydrogen, a C₁-C₆ alkyl group, a C₆-C₁2 aryl group, or a C₇-C₁₈ aralkyl group; R₂ represents hydrogen, a C₁-C₆ alkyl group or a halogen atom; and A is represented by 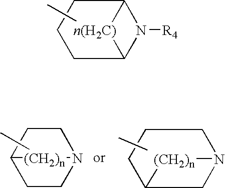
where n is 2 or 3; and R₄ represents a C₁-C₆ alkyl group.

In some embodiments, compounds having 5-HT₃ receptor agonist activity are represented by Formula I, wherein R₁ represents hydrogen or a C₁-C₃ alkyl group, R₂ represents hydrogen, a C₁-C₃ alkyl group or a halogen atom, R₃ represents hydrogen, R₄ represents a C₁-C₃ alkyl group and n is an integer chosen from 2 or 3.

In yet other embodiments, a compound having 5-HT₃ receptor agonist activity is represented by structural Formula shown below (Formula II): 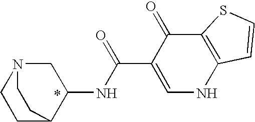
or a pharmaceutically acceptable salt, solvate or hydrate thereof.

In some embodiments, the compound of Formula II has the (R) configuration at the chiral carbon atom which is designated with an asterisk (*). The chemical name of the compound set forth in Formula II having the (R) configuration at the designated chiral carbon is: (R)-N-1-azabicyclo[2.2.2]oct-3-yl-4,7-dihydro-7-oxothieno[3,2-b]pyridine-6-carboxamide. When the compound is in the form of the monohydrochloride, it is known as MKC-733 (or DDP733) and pumosetrag (CAS Number: 194093-42-0). When the compound of Formula II has the (S) configuration at the chiral carbon atom designated with an asterisk (*), the chemical name is (S)-N-1-azabicyclo[2.2.2]oct-3-yl-4,7-dihydro-7-oxothieno[3,2-b]pyridine-6-carboxamide.

It is understood that structural Formula II includes the tautomeric form depicted by Formula IIA below:

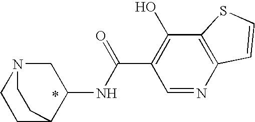

Likewise, it is understood that Formula IIA includes the tautomeric form represented by Formula II.

For example, when Formula II has the (R) configuration at the designated chiral carbon the compound is referred to as: (R)-N-1-azabicyclo[2.2.2]oct-3-yl-4,7-dihydro-7-oxothieno[3,2-b]pyridine-6-carboxamide which is understood to include the tautomeric form: (R)-N-1-azabicyclo[2.2.2]oct-3-yl)-7-hydroxythieno[3,2-b]pyridine-6-carboxamide.

Likewise, when Formula IIA has the (R) configuration at the designated chiral carbon the compound is referred to as: (R)-N-1-azabicyclo[2.2.2]oct-3-yl )-7-hydroxythieno[3,2-b]pyridine-6-carboxamide, which is understood to include the tautomeric form: (R)-N-1-azabicyclo[2.2.2]oct-3-yl-4,7-dihydro-7-oxothieno[3,2-b]pyridine-6-carboxamide.

In some embodiments, compounds having 5-HT₃ receptor agonist activity are represented by Formula III below or a pharmaceutically acceptable salt, solvate or hydrate thereof: 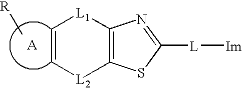
where R represents hydrogen, halogen, hydroxyl, a C₁-C₆ alkoxy group, a carboxy group, a C₁-C₆ alkoxycarbonyl group, a nitro group, an amino group, a cyano group or protected hydroxyl; and 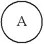
is a phenyl ring or a naphthalene ring; L is a direct bond or a C₁-C₆ alkylene group; L₁ and L₂ are defined so that one is a direct bond and the other is chosen from: (a) a C₁-C₆ alkylene group optionally containing an interrupting oxygen or sulfur atom therein; (b) an oxygen atom or sulfur atom; and (c) a C₁-C₆ alkenylene group. Im represents a group having the formula below 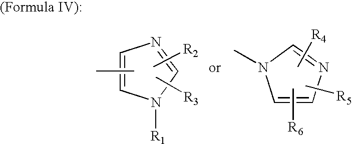
where R₁-R₆ are the same or different each representing hydrogen or a C₁-C₆ alkyl group.

In yet other embodiments, in the compound according to Formula III, 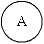
is a phenyl ring, L₁ is a direct bond and L₂ is an alkylene group or an alkenylene group.

In still other embodiments, a compound having 5-HT₃ receptor agonist activity is represented by structural Formula below (Formula V): 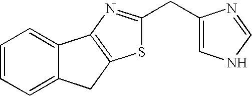
or a pharmaceutically acceptable salt, solvate, or hydrate thereof. This compound is commonly referred to in the art as YM 31636. The chemical name of the compound set forth in Formula V is: 2-(1H-imidazol-4-ylmethyl)-8H-indeno[1,2-d]thiazole. The compound YM 31636 and derivatives thereof, are also described in U.S. Pat. No. 5,834,499, the entire content of which is incorporated by reference herein.

In some embodiments, a method of treating a GI motility disorder in a subject includes administering a first compound having 5-HT₃ receptor agonist activity and at least one second compound, where the combination of the first and at least one second compound results in an increase in GI motility either by increasing colonic propulsion and/or increasing GI secretions. Such an increase can either be additive or synergistic, compared to the effect on GI motility in the presence of one of the first compound or the at least one second compound when given alone. In some embodiments, administration of a first compound having 5-HT₃ receptor agonist activity and at least one second compound results in a synergistic increase in GI motility. In some embodiments, one advantage of the compositions described herein is that at least one detrimental side effect associated with single administration of the first compound having 5-HT₃ receptor agonist activity or at least one second compound, as described herein, is alleviated or decreased by concurrent administration of the first and at least one second compounds. One reason for this advantage is that less of each compound is needed in a combination, relative to the amount required when each is used alone. Furthermore, normal amounts of each compound when given in combination could provide for greater efficacy in subjects who are either unresponsive or minimally responsive to each compound when used alone. An added benefit of such compositions is that they alleviate visceral pain associated with GI hypomotility disorders such as IBS with constipation.

In some embodiments, methods described herein include administering a first compound having 5-HT₃ receptor agonist activity and at least one second compound chosen from: compounds having 5-HT₄ receptor agonist activity (e.g., tegaserod, cisapride, prucalopride, SL 65.0155, ATI-7505, and TD-2749); compounds having both 5-HT₄ receptor agonist activity and 5-HT₃ receptor antagonist activity (e.g., mosapride, renzapride, and E-3620); compounds having both 5-HT₄ receptor agonist activity and dopamine receptor antagonist activity (e.g., metoclopramide); compounds having both 5-HT₄ receptor agonist activity and D₂-receptor antagonist activity (e.g., itopride); compounds having D₂-receptor antagonist activity (e.g., chlorpromazine, prochlorperazine, haloperidol, and alizapride); compounds having motilin receptor agonist activity (e.g., erythromycin, mitemcinal, and atilmotin); compounds having GABA-B receptor agonist activity (e.g., baclofen and XP-19986); compounds having corticotropin-releasing factor (CRF-1) receptor antagonist activity (e.g., CP-316311, TS-041, 876008, ONO-2333MS, MG-561, and NBI-34041); compounds having opioid receptor antagonist activity (e.g., naltrexone, PTI-901, alvimopan, methylnaltrexone, and naloxone); compounds having alpha 2 adrenoreceptor agonist activity (e.g., clonidine, tizanidine, and guanfacine); compounds having tachykinin (NK₁, NK₂, and/or NK₃) receptor antagonist activity (e.g., nepadutant, saredutant, talnetant, and osanetant); compounds having muscarinic cholinergic receptor agonist activity (e.g. bethanechol and carbachol); acetylcholinesterases inhibitors (e.g., neostigmine); compounds having mixed serotonin and noradrenaline reuptake inhibitor activity (e.g., milnacipran, venlafaxine, desvenlafaxine, sibutramine, and duloxetine); benzodiazepine-like molecules (e.g., dextofisopam, levotofisopam, diazepam, lorazepam, alprazolam, and clonazepam); compounds having nitric oxide synthase (NOS) inhibitory activity (e.g., SC-81490, targinine, and 274150); cannabinoid receptor modulators (e.g., dronabinol, nabilone, rimonabant, cannabidiol, and SAB-378); compounds having reversible cholecystokinin 1 (CCK1) receptor antagonist activity (e.g., dexloxiglumide (CR 2017), and devazepide; compounds that activate chloride channels (e.g. lubiprostone); compounds having guanylate cyclase receptor agonist activity (e.g., MD-1100); and glucagon-like peptide (GLP) analogs (e.g. GTP-010).

In some embodiments, methods described herein include administering a first compound having 5-HT₃ receptor agonist activity (e.g., DDP733) and at least one second compound having opioid receptor antagonist activity (e.g., naltrexone and alvimopan). Examples of opioid receptor antagonists which may be used in the compositions and methods featured by this invention can be found in U.S. Pat. Nos. 5,250,542, 5,434,171, 6,051,806 and 6,469,030, the entire contents of which are incorporated by reference herein.

In a particular embodiment, methods featured herein include administering a combination of DDP733 and naltrexone for treating a gastrointestinal hypomotility disorder, such as, for example, constipation (e.g., chronic idiopathic constipation and acute constipation), irritable bowel syndrome with constipation (IBS-c), and post operative ileus. In another embodiment, methods featured herein include administering a combination of DDP733 and alvimopan for treating a gastrointestinal hypomotility disorder.

Table I below provides the CAS numbers and chemical formulae for the various compounds listed above.

TABLE I

| | | CAS | Chemical Name/ | CAS Numbers Of |
|---|---|---|---|---|
| Compound | Activity | Numbers | Molecular Formula | Related Compounds |
| Tegaserod | 5-HT₄ receptor | 189188-57-6 | 3-(5-Methoxy-1H-indol-3-methylene)-N- | 145158-71-0 (free base) |
| Maleate | agonist | | pentylcarbazimidamide hydrogen maleate | |
| | | | C₁₆H₂₃N₅O.C₄H₄O₄ | |
| Cisapride | 5-HT₄ receptor | 81098-60-4 | (±)-cis-4-amino-5-chloro-N-[1-[3-(4- | |
| Hydrate | agonist | | fluorophenoxy)propyl]-3-methoxy-4-piperidinyl]- | |
| | | | 2-methoxybenxamide monohydrate | |
| | | | C₂₃H₂₉ClFN₃O₄.H₂O | |
| Prucalopride | 5-HT4 receptor | 179474-81-8 | 4-Amino-5-chloro-2,3-dihydro-N-(1-(3 methoxypropyl)-4- | 179474-80-7 |
| | agonist | | piperidyl)-7-benzofurancarboxamide | (monohydrochloride) |
| | | | C₁₈H₂₆ClN₃O₃ | |
| ATI-7505 | 5-HT4 receptor | Unknown | Unknown | |
| | agonist | | | |
| TD-2749 | 5-HT4 receptor | Unknown | Unknown | |
| | agonist | | | |
| Mosapride | 5-HT₄ receptor | 112885-42-4 | (+−)-4-Amino-5-chloro-2-ethoxy-N-((4-(4- | 112885-41-3 (anhydrous |
| Citrate | agonist and | | fluorobenzyl)-2-morpholinyl)methyl)benzamide citrate | free base non-specified |
| | 5-HT₃ receptor | | C₂₁H₂₅ClFN₃O₃.C₆H₈O₇ | stereoch.) |
| | antagonist | | | 112885-43-5 (monoHCl salt |
| | | | | anhydrous non-specified |
| | | | | stereoch.) |
| | | | | 131322-37-7 (HCl salt |
| | | | | anhydrous non-specified |
| | | | | stereoch.) |
| | | | | 144256-27-9 (anhydrous |
| | | | | free base) |
| Renzapride | 5-HT₄ receptor | 109872-41-5 | 4-Amino-5-chloro-2-methoxy-N-(1-azabicyclo-(3.3.1)- | 88721-77-1 (free base; |
| Hydrochloride | agonist and | | non-4-yl)benzamide monohydrochloride | no stereoch.) |
| | 5-HT₃ receptor | | C₁₆H₂₂ClN₃O₂.HCl | 112727-80-7 (free base) |
| | antagonist | | | |
| E-3620 | 5-HT₄ receptor | Unknown | (−)-4-Amino-5-chloro-N-(endo-8-methyl-8- | |
| | agonist and | | azabicyclo[3.2.1]oct-3alpha-yl)-2-[1(S)- | |
| | 5-HT₃ receptor | | methyl-2-butynyloxy]benzamide monohydrochloride | |
| | antagonist | | C₂₀H₂₇Cl₂N₃O₂ | |
| Metoclopramide | 5-HT₄ receptor | 54143-57-6 | 4-Amino-5-chloro-N-[2-(diethylamino)ethyl]-2- | 364-62-5 (anhydrous free |
| Mono-hydrochloride | agonist and | | methoxybenzamide monohydrochloride monohydrate | base) |
| monohydrate | dopamine | | C₁₄H₂₂ClN₃O₂.HCl.H₂O | 7232-21-5 (anhydrous) |
| | receptor | | | 2576-84-3 (dihydrochloride |
| | antagonist | | | monohydrate) |
| | | | | 5581-45-3 (dihydrochloride |
| | | | | monohydrate) |
| | | | | 102670-58-6 (sulfide) |
| | | | | 139339-72-3 (sulfone) |
| Itopride | 5-HT₄ receptor | 122892-31-3 | N-((4-(2-(Dimethylamino)ethoxy)phenyl)methyl)- | 122898-67-3 (free base) |
| hydrochloride | agonist and | | 3,4-dimethoxybenzamide monohydrochloride | |
| | D₂ receptor | | C₂₀H₂₆N₂O₄.HCl | |
| | antagonist | | | |
| Chlorpromazine | D₂ receptor | 69-09-0 | 10-(3-dimethylaminopropyl)-2-chlorophenothiazine | |
| hydrochloride | antagonist | | monohydrochloride | |
| (oral and | | | C₁₇H₁₉ClN₂S.Cl—H | |
| injectable | | | | |
| forms) | | | | |
| Chlorpromazine | D₂ receptor | 50-53-3 | 10-(3-dimethylaminopropyl)-2-chlorophenothiazine | 969-99-3 (sulfoxide) |
| (suppositories) | antagonist | | C₁₇H₁₉ClN₂S | 4337-86-4 (sulfone) |
| Prochlorpera- | D₂ receptor | 84-02-6 | 2-chloro-10-[3-(4-methyl-1-piperazinyl) | 58-38-8 (free base)- |
| zine Maleate | antagonist | | propyl]-10H-phenothiazine(Z)-2-butenedioate(1:2) | suppositories |
| | | | C₂₀H₂₄ClN₃S.2C₄H₄O₄ | 1257-78-9 (edisylate)- |
| | | | | vials, syrup |
| Haloperidol | D₂ receptor | 52-86-8 | 4-[4-(p-chlorophenyl)-4-hydroxypiperidino]- | 74050-97-8 (decanoate) |
| | antagonist | | 4′-fluorobutyrophenone | 1511-16-6 (hydrochloride) |
| | | | C₂₁H₂₃ClFNO₂ | 75478-79-4 (lactate) |
| Alizapride | D₂ receptor | 59338-93-1 | 6-Methoxy-N-((1-(2-propenyl)-2-pyrrolidinyl)methyl)- | 59338-87-3 (hydrochloride) |
| | antagonist | | 1H-benzotriazole-5-carboxamide | |
| | | | C₁₆H₂₁N₅O₂ | |
| Baclofen | (GABA)-B | 1134-47-0 | 4-amino-3-(4-chlorophenyl)-butanoic acid | |
| | receptor | | C₁₀H₁₂ClNO₂ | |
| | agonist | | | |
| XP-19986 | (GABA)-B | Unknown | Unknown | |
| | receptor | | | |
| | agonist | | | |
| Erythromycin | Motilin | 114-07-8 | (3R*,4S*,5S*,6R*,7R*,9R*,11R*,12R*,13S*,14R*)- | 96128-89-1 (acistrate) |
| | receptor | | 4-[(2,6-dideoxy-3-C-methyl-3-O-methyl-a-L-ribo- | 3521-62-8 (estolate) |
| | agonist | | hexopyranosyl)oxy]-14-ethyl-7,12,13-trihydroxy- | 1264-62-6 (ethylsuccinate) |
| | | | 3,5,7,9,11,13-hexamethyl-6-[[3,4,6-trideoxy- | 23067-13-2 (glucoheptonate) |
| | | | 3-(dimethylamino)-β-D-xylo- | 3847-29-8 (lactobionate) |
| | | | hexopyranosyl]oxy]oxacyclotetradecane-2,10-dione | 134-36-1 (propionate) |
| | | | C₃₇H₆₇NO₁₃ | 643-22-1 (stearate) |
| | | | | 84252-03-9 (stinoprate) |
| Mitemcinal | Motilin | 154802-96-7 | C₄₄H₇₃NO₁₆ | 154738-42-8 (free base) |
| Fumarate | receptor | | | |
| | agonist | | | |
| Atilmotin | Motilin | 533927-56-9 | N,N,N-Trimethyl-L-phenylalanyl-L-valyl-L-prolyl-L- | |
| | receptor | | isoleucyl-L-phenylalanyl-L-threonyl-L-tyrosyl- | |
| | agonist | | glycyl-L-glutamyl-L-leucyl-L-glutaminyl-D-arginyl- | |
| | | | L-leucyl-L-lysinamide | |
| | | | C₈₆H₁₃₄N₂₀O₁₉ | |
| Naltrexone | Opioid | 16676-29-2 | 17-(Cyclopropylmethyl)-4,5-alpha-epoxy-3,14- | 16590-41-3 (free base) |
| Hydrochloride | receptor | | dihydroxy-morphinan-6-one hydrochloride | |
| | antagonist | | C₂₀H₂₃NO_{4.}HCl | |
| PTI-901 (oral | Opioid | Unknown | Unknown | |
| low | receptor | | | |
| dose | antagonist | | | |
| formulation of | | | | |
| naltrexone) | | | | |
| Alvimopan | Opioid | 170098-38-1 | (((2S)-2-(((3R,4R)-4-(3-hydroxyphenyl)-3,4- | |
| Hydrate | receptor | | dimethylpiperidin-1-yl)methyl)-3- | |
| | antagonist | | phenylpropanoyl)amino)acetic acid dihydrate | |
| | | | C₂₅H₃₂N₂O₄.2H₂O | |
| Methylnaltre- | Opioid | 73232-52-7 | (5alpha)-17-(cyclopropylmethyl)-4,5-epoxy-3,14- | 73232-53-8 (iodide) |
| xone Bromide | receptor | | dihydroxy-17-methyl-6-oxomorphinanium bromide | 83387-25-1 (cation) |
| | antagonist | | C₂₁H₂₆BrNO₄ | 125292-47-9 (racemate) |
| Naloxone | Opioid | 357-08-4 | (−)-17-Allyl-4, 5α-epoxy-3,14- | 465-65-6 (free base) |
| Hydrochloride | receptor | | dihydroxymorphinan-6-one hydrochloride | 51481-60-8 (dihydrate) |
| | antagonist | | C₁₉H₂₁NO₄.ClH | |
| Neostigmine | Cholinergic | 114-80-7 | 3-(((dimethylamino)carbonyl)oxy)-N,N,N- | 59-99-4 (free base) |
| bromide | receptor | | trimethylbenzenaminium bromide | |
| | agonist | | C₁₂H₁₉BrN₂O₂ | |
| Neostigmine | Cholinergic | 51-60-5 | 3-(((dimethylamino)carbonyl)oxy)-N,N,N- | 59-99-4 (free base) |
| Methylsulfate | receptor | | trimethylbenzenaminium methyl sulfate | |
| | agonist | | C₁₃H₂₂N₂O₆S | |
| Carbachol | Cholinergic | 51-83-2 | 2-[(aminocarbonyl)oxy]-N,N,N- | |
| | receptor | | trimethylethanaminium chloride | |
| | agonist | | C₆H₁₅ClN₂O₂ | |
| Bethanechol | Acetylcholines- | 590-63-6 | 2-((Aminocarbonyl)oxy)-N,N,N-trimethyl- | 674-38-4 (free base) |
| Chloride | terase | | 1-propanaminium chloride | |
| | | | C₇H₁₇ClN₂O₂ | |
| Milnacipran | SNRI | 101152-94-7 | (1R,2S)-rel-2-(Aminomethyl)-N,N-diethyl-1- | 92623-85-3 (free base) |
| Hydrochloride | | | phenylcyclo-propane carboxamide | 86181-08-0 (cis-isomer) |
| | | | monohydrochloride | |
| | | | C₁₅H₂₂N₂O.HCl | |
| Venlafaxine | SNRI | 99300-78-4 | (R/S)-1-[2-(dimethylamino)-1-(4- | 93413-69-5 (free base) |
| Hydrochloride | | | methoxyphenyl)ethyl]cyclohexanol | |
| | | | hydrochloride | |
| | | | OR | |
| | | | (±)-1-[α-[(dimethylamino)methyl]-p- | |
| | | | methoxybenzyl]cyclohexanol hydrochloride | |
| | | | C₁₇H₂₇NO₂.HCl. | |
| Desvenlafaxine | SNRI | 386750-22-7 | 4-(2-(dimethylamino)-1-(1- | 93413-62-8 (free base, |
| Succinate | | | hydroxycyclohexyl)ethyl)phenol succinate | anhydrous) |
| | | | hydrate | 448904-47-0 (anhydrous) |
| | | | C₂₀H₃₃NO₇ | 448904-48-1 (hemisuccinate) |
| Sibutramine | SNRI | 125494-59-9 | (+−)-1-(4-chlorophenyl)-N,N-dimethyl- | 106650-56-0 (free base) |
| hydrochloride | | | alpha-(2-methylpropyl)cyclobutanemethanamine | |
| monohydrate | | | hydrochloride monohydrate | |
| | | | C₁₇H₂₆ClN.HCl.H₂O | |
| Duloxetine | SNRI | 136434-34-9 | (+)-(S)-N-methyl-γ-(1-naphthyloxy)-2- | 116539-59-4 (free base) |
| Hydrochloride | | | thiophenepropylamine hydrochloride | 116817-77-7 (oxalate salt) |
| | | | C₁₈H₁₉NOS.HCl | 116817-86-8 (maleate) |
| Dextofisopam | Benzodiazepine | 82059-50-5 | (+)-1-(3,4-Dimethoxyphenyl)-5(R)-ethyl- | |
| | | | 7,8-dimethoxy-4-methyl-5H-2,3-benzodiazepine | |
| | | | C₂₂H₂₆N₂O₄ | |
| Levotofisopam | Benzodiazepine | 82059-51-6 | 1-(3,4-Dimethoxyphenyl)-5(S)-ethyl-7,8-dimethoxy- | |
| | | | 4-methyl-5H-2,3-benzodiazepine | |
| | | | C₂₂H₂₆N₂O₄ | |
| Diazepam | Benzodiazepine | 439-14-5 | 7-Chloro-1-methyl-5-phenyl-1,3-dihydro-1,4- | |
| | | | benzodiazepin-2-one | |
| | | | C₁₆H₁₃ClN₂O | |
| Lorazepam | Benzodiazepine | 846-49-1 | 7-chloro-5-(o-chlorophenyl)-1,3-dihydro-3-hydroxy- | |
| | | | 2H-1,4-benzodiazepin-2-one | |
| | | | C₁₅H₁₀Cl₂N₂O₂ | |
| Clonazepam | Benzodiazepine | 1622-61-3 | 5-(2-chlorophenyl)-1,3-dihydro-7-nitro-2H-1,4- | |
| | | | benzodiazepin-2-one | |
| | | | C₁₅H₁₀ClN₃O₃ | |
| Alprazolam | Benzodiazepine | 28981-97-7 | 8-Chloro-1-methyl-6-phenyl-4H-s- | |
| | | | triazolo[4,3-α][1,4]benzodiazepine | |
| | | | C₁₇H₁₃ClN₄ | |
| SC-81490 | Nitric oxide | Unknown | Unknown | |
| | synthase | | | |
| | Inhibitor | | | |
| Targinine | Nitric oxide | 53308-83-1 | N5-(Methylamidino)-L-ornithine monoacetate | |
| acetate | synthase | | C₉H₂₀N₄O₄ | |
| | Inhibitor | | | |
| 274150 | Nitric oxide | 210354-22-6 | C₈H₁₇N₃O₂S | 210354-23-7 (undefined |
| | synthase | | | isomer) |
| | Inhibitor | | | |
| Dronabinol | Cannabinoid | 1972-08-3 | (6aR,10aR)-6,6,9-trimethyl-3-pentyl-6a,7,8,10a- | |
| | modulator | | tetrahydro-6H-Dibenzo(b,d)pyran-1-ol | |
| | | | C₂₁H₃₀O₂ | |
| Nabilone | Cannabinoid | 51022-71-0 | (+−)-trans-6,6-Dimethyl-3-(1,1- | |
| | modulator | | dimethylheptyl)--1-hydroxy-6,6a,7,8,10,10a- | |
| | | | hexahydro-9H-Dibenzo(b,d)pyran-9-one | |
| | | | C₂₄H₃₆O₃ | |
| Rimonabant | Cannabinoid | 158681-13-1 | 5-(4-Chlorophenyl)-1-(2,4-dichlorophenyl)- | 168273-06-1 (free base) |
| hydrochloride | Modulator | | 4-methyl-N-1-piperidinyl-1H-pyrazole-3- | |
| | | | carboxamide monohydrochloride | |
| | | | C₂₂H₂₁Cl₃N₄O.Cl—H | |
| SAB-378 | Cannabinoid | Unknown | Unknown | |
| | modulator | | | |
| Cannabidiol | Cannabinoid | 13956-29-1 | (1R-trans)-2-(3-methyl-6-(1-methylethenyl)- | |
| | modulator | | 2-cyclohexen-1-yl)-5-pentyl-1,3-benzenediol | |
| | | | C₂₁H₃₀O₂ | |
| Dexloxiglumide | Cholecysto- | 119817-90-2 | (R)-4-(3,4-Dichlorobenzamido)-N-(3- | |
| | kinin | | methoxypropyl)-N-pentylglutaramic acid | |
| | antagonist | | C₂₁H₃₀Cl₂N₂O₅ | |
| Devazepide | Cholecysto- | 103420-77-5 | 3(S)-(−)-1,3-Dihydro-3-(2- | |
| | kinin | | indolecarbonylamino)-1-methyl-5-phenyl-2H-(1,4) | |
| | antagonist | | benzodiazepin-2-one | |
| | | | C₂₅H₂₀N₄O₂ | |
| Lubiprostone | Chloride | 333963-40-9 | (−)-7-((2R,4aR,5R,7aR)-2-(1,1-Difluoropentyl)- | |
| | channel | | 2-hydroxy-6-oxooctahydrocyclopenta(b)pyran-5- | |
| | activator | | yl)heptanoic acid | |
| | | | C₂₀H₃₂F₂O₅ | |
| MD-1100 | Guanylate | 742095-77-8 | L-Cysteinyl-L-cysteinyl-L-glutamyl-L-tyrosyl-L- | |
| | cyclase C | | cysteinyl-L-cysteinyl-L-asparaginyl-L-prolyl-L- | |
| | receptor | | alanyl-L-cysteinyl-L-threonyl-glycyl-L-cysteinyl- | |
| | agonist | | L-tyrosine | |
| | | | C₅₉H₈₅N₁₅O₂₁S₆ | |
| CP-316311 | CRF receptor | Unknown | Unknown | |
| | antagonist | | | |
| TS-041 | CRF receptor | Unknown | Unknown | |
| | antagonist | | | |
| 876008 | CRF receptor | Unknown | Unknown | |
| | antagonist | | | |
| ONO-2333MS | CRF receptor | Unknown | Unknown | |
| | antagonist | | | |
| AAG-561 | CRF receptor | Unknown | Unknown | |
| | antagonist | | | |
| NBI-34041 | CRF receptor | Unknown | Unknown | |
| | antagonist | | | |
| Clonidine | Alpha2 | 4205-91-8 | 2-(2,6-dichlorophenylamino)-2-imidazoline | 4205-90-7 (free base) |
| Hydrochloride | receptor | | hydrochloride | |
| | agonist | | C₉H₉Cl₂N₃.HCl | |
| Tizanidine | Alpha2 | 64461-82-1 | 5-chloro-4-(2-imidazolin-2-ylamino)-2,1,3- | 51322-75-9 (free base) |
| Hydrochloride | receptor | | benzothiodiazole hydrochloride | |
| | agonist | | C₉H₈ClN₅S.HCl | |
| Guanfacine | Alpha2 | 29110-48-3 | N-Amidino-2-(2,6-dichlorophenyl)acetamide | 29110-47-2 (free base) |
| hydrochloride | receptor | | Monohydrochloride | |
| | agonist | | C₉H₉Cl₂N₃O.HCl | |
| Nepadutant | NK receptor | 183747-35-5 | Cyclo(N-(2-acetamido-2-deoxy-beta-D- | |
| | antagonist | | glucopyranosyl)-L-asparginyl-L-alpha-aspartyl- | |
| | | | L-tryptophyl-L-phenylalanyl-L-2,3- | |
| | | | diaminopropionyl-L-leucyl),cyclic(2-5)-peptide | |
| | | | C₄₅H₅₈N₁₀O₁₃ | |
| Saredutant | NK receptor | 142001-63-6 | (S)-N-(4-(4-(acetylamino)-4-phenyl-1-piperidinyl)- | |
| | antagonist | | 2-(3,4-dichlorophenyl)butyl)-N-methylbenzamide | |
| | | | C₃₁H₃₅Cl₂N₃O₂ | |
| Talnetant | NK receptor | 174636-32-9 | (S)-3-hydroxy-2-phenyl-N-(1-phenylpropyl)4- | 174636-26-1 (undefined |
| | antagonist | | Quinolinecarboxamide | isomer) |
| | | | C₂₅H₂₂N₂O₂ | 174636-33-0 ((R)-isomer) |
| | | | | 204519-66-4 (monoHCl) |
| Osanetant | NK receptor | 160492-56-8 | (R)-N-(1-(3-(1-benzoyl-3-(3,4-dichlorophenyl)- | 173050-51-6 (monoHCl) |
| | antagonist | | 3-piperidinyl)propyl)-4-phenyl-4-piperidinyl)- | |
| | | | N-methylacetamide | |
| | | | C₃₅H₄₁Cl₂N₃O₂ | |
| GTP-010 | GLP analog | Unknown | Unknown | |

In some embodiments, a first compound having a 5-HT₃ receptor agonist activity and at least one second compound are included in a single composition, which is administered to a subject having GI hypomotility. In other embodiments, a first compound having a 5HT₃ receptor agonist activity and at least one second compound are administered separately to such a subject. The first and at least one second compound may either be co-administered to a subject (i.e., at the same time) or be administered sequentially (i.e., one after the other). The phrase “a disorder associated with GI hypomotility,” as used herein, refers to disorders of the GI tract where the normal orderly movement of ingested material through the GI tract is impaired. The phrase “a disorder associated with GI hypomotility,” includes disorders of which GI hypomotility is a symptom and also disorders of which GI motility is a cause. In some embodiments, abnormal GI motility is a component of such a disorder, for example, in case of a multi-component GI disorder. Examples of such disorders include, for example, gastroesophageal reflux disease (GERD), nocturnal gastroesophageal reflux disease (n-GERD); dyspepsia, constipation including slow transit constipation, normal transit constipation, acute constipation, chronic idiopathic constipation, constipation associated with irritable bowel syndrome and constipation due to increased tone of the large intestine, ileus and post-operative ileus, narcotic bowel syndrome, gastroparesis including diabetic gastroparesis and intestinal pseudo-obstruction gastroparesis, Hirschsprung's disease, decreased peristalsis of the esophagus and/or stomach and/or the small and/or the large intestine, esophagitis, non-ulcer dyspepsia, pseudo-obstruction of the bowels and/or the colon, impaired colonic transit, epigastric pain, postoperative gut atony, recurrent nausea and vomiting, anorexia nervosa, dyskinesias of the biliary system, tachygastria and problems of gastric emptying due to tachygastria, intestinal spasms and cramps, irritable bowel syndrome with diarrhea, peptic ulcer diseases, inflammatory diseases of the stomach and bowel including gastritis, inflammatory bowel disease, duodenitis, intestinal and post-operative intestinal atony, and drug-induced delayed transit.

Gastroparesis is the delayed emptying of stomach contents into the duodenum. Symptoms of gastroparesis include, for example, stomach upset, heartburn, nausea and vomiting. Gastroparesis can be brought about by an abnormality in the stomach or as a complication of diseases such as diabetes, progressive systemic sclerosis, anorexia nervosa and myotonic dystrophy. Constipation can result from conditions such as reduced intestinal muscle tone or intestinal spasticity. Post-operative ileus is an obstruction or a kinetic impairment in the intestine due to a disruption in muscle tone following surgery. Dyspepsia is an impairment of the function of digestion that can arise from functional or organic causes. It is understood that compositions of the present invention can be used either to treat the actual cause of the disorder or condition being treated and/or to provide relief to the patients from symptoms of a disorder.

Compositions described herein can be prepared, for example, by combining effective amounts of a first compound having 5-HT₃ receptor agonist activity and at least one second compound, with a pharmaceutically acceptable carrier, which may take a wide variety of forms depending on the form of preparation desired for administration. An example of a pharmaceutically acceptable carrier used in the compositions described herein is DMSO, for example, 0.1% DMSO.

In some embodiments, pharmaceutical compositions can be in unitary dosage form suitable for administration orally, rectally or by parenteral injection. For example, in preparing compositions in oral dosage form, any of the usual pharmaceutical media may be employed, such as, for example, water, glycols, oils, alcohols and the like, as in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are employed. For parenteral compositions, carriers usually comprise sterile water, at least in large part, though other ingredients, for example, to aid solubility, may be included. Injectable solutions, for example, are prepared using a carrier which comprises saline solution, glucose solution or a mixture of saline and glucose solution. Injectable suspensions may also be prepared in which case appropriate liquid carriers, suspending agents and the like may be employed. In case of compositions suitable for percutaneous administration, carrier optionally comprises a penetration enhancing agent and/or a suitable wetting agent, which may be combined with suitable additives of any nature in minor proportions, which additives do not cause a significant deleterious effect to the skin. Additives may facilitate the administration to the skin and/or may be helpful for preparing desired compositions. These compositions may be administered in various ways, e.g., as a transdermal patch, as a spot-on, as an ointment.

It is especially advantageous to formulate the pharmaceutical compositions described herein in dosage unit form for ease of administration and uniformity of dosage. Dosage unit form, as used herein, refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

In general it is contemplated that a therapeutically effective amount of a first or a second compound would be from about 0.0001 mg/Kg to 0.001 mg/Kg; 0.001 mg/kg to about 10 mg/kg body weight or from about 0.02 mg/kg to about 5 mg/kg body weight. In some embodiments, a therapeutically effective amount of a first or a second compound is from about 0.007 mg to about 0.07 mg, about 0.07 mg to about 700 mg, or from about 1.4 mg to about 350 mg. A method of prophylactic or curative treatment may also include administering the composition in a regimen of between one to five intakes per day.

In some embodiments, a therapeutically effective amount of a first compound or a second compound includes but is not limited to the amount less than about 0.01 mg/dose, or less than about 0.5 mg/dose, or less than about 1 mg/dose, or less than about 2 mg/dose, or less than about 5 mg/dose, or less than about 10 mg/dose, or less than about 20 mg/dose, or less than about 25 mg/dose, or less than about 50 mg/dose, or less than about 100 mg/dose. The number of times a day a first or a second compound is administrated to a subject can be determined based on various criteria commonly used in the art and/or those described herein.

Various clinical tests can be used for assaying the effect of compositions described herein on GI motility, for example, by measuring the rate of gastric emptying following administration of a composition described herein and comparing it to the rate following administration of a single compound in the composition. For example, gastric emptying scintigraphy of a radiolabeled solid meal is an accepted method to test for delayed gastric emptying. Conventionally, the test is performed for 2 hours after ingestion of a radiolabeled meal. For the test meal preparation, the radioisotope is cooked into the solid portion of the meal. Additionally, breath testing can be used to measure gastric emptying using the nonradioactive isotope ¹³C to label octanoate, a medium-chain triglyceride, which can be incorporated into a solid meal. Studies have also reported labeling the proteinaceous algae (Spirulina) with ¹³C. By measuring ¹³C in breath samples, gastric emptying can be indirectly determined. It is understood that any method described herein or known in the art can be used for measuring an effect of compositions described herein on GI motility including, for example, the colonic pellet propulsion test discussed in the Examples. In addition, gastrointestinal motility may be measured in humans by using radiopaque markers such as described in Horikawa et al. Scand J Gastroenterol., 34(12):1190-1195 (1999), incorporated by reference herein. Another method which can be used is to examine inhibition of pharmacologically-induced constipation in a mammal (e.g., constipation induced by clonidine, morphine or loperamide) as described in Saito et al., Japanese J. Pharmacol. 89(2):133-141 (2002), incorporated by reference herein.

A combination of compounds described herein can either result in synergistic increase in GI motility, relative to motility following administration of each compound when used alone, or such an increase can be additive. Compositions described herein typically include lower dosages of each compound in a composition, thereby avoiding adverse interactions between compounds and/or harmful side effects, such as ones which have been reported for similar compounds. Furthermore, normal amounts of each compound when given in combination could provide for greater efficacy in subjects who are either unresponsive or minimally responsive to each compound when used alone.

A synergistic effect can be calculated, for example, using suitable methods such as, for example, the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

In some embodiments, increasing GI motility comprises increasing esophageal motility. As used herein, “increasing esophageal motility” refers to increasing peristaltic wave frequency and/or peristaltic wave amplitude. In other embodiments increasing GI motility comprises increasing velocity of colonic propulsion, as described herein. Increasing esophageal motility may also be evidenced by decreasing the duration for which the lower esophagus is exposed to potentially harmful pH levels (<pH 4), an increase in lower esophageal sphincter pressure (LESP) and/or a decrease in number or frequency of transient lower esophageal sphincter relaxations (tLESR's).

As discussed above, an advantage of the compositions described herein is the ability to use less of each compound than is needed when each is administered alone. Another advantage is that greater efficacy may be achieved in subjects who are either unresponsive or minimally responsive to each compound when used alone in normal amounts by giving the agents in combination. As such, undesirable side effects associated with the compounds are reduced (partially or completely) and/or improved efficacy may be achieved. A reduction in side effects with or without improved efficacy can result in increased patient compliance over current treatments. An added benefit of such compositions is that they would alleviate visceral pain associated with GI hypomotility disorders such as irritable bowel syndrome.

As used herein, “therapeutically effective amount” refers to an amount of a first compound and at least one second compound, as used herein, sufficient to elicit a desired biological response. In case of the methods described herein, a desired biological response is a reduction (complete or partial) of at least one symptom associated with the disorder being treated and/or improved efficacy. As with any treatment, particularly treatment of a multi-symptom disorder, for example, GERD, it is advantageous to treat as many disorder-related symptoms as the patient experiences. The phrase “therapeutically effective amount” encompasses amounts of a first compound having 5-HT₃ receptor agonist activity (or a pharmaceutically acceptable salt, hydrate or solvate thereof) and at least one second compound, as described herein, wherein the combination of the first and at least one second compound results in an increase in GI motility. Any amounts of a first compound having 5-HT₃ receptor agonist activity (or a pharmaceutically acceptable salt, hydrate or solvate thereof) and at least one second compound can be used in the prevention, treatment, and/or management of a disorder, as described herein, provided that the combination of the first and at least one second compound results in an increase in GI motility, e.g., by promoting physical propulsions anywhere in the GI tract and/or increasing GI secretions.

The term “subject,” as used herein, refers to animals such as mammals, including, but not limited to, primates (e.g., humans), cows, sheep, goats, horses, pigs, dogs, cats, rabbits, guinea pigs, rats, mice or other bovine, ovine, equine, canine, feline, rodent or murine species.

As used herein, “treat,” “treating” and “treatment” refer to a reduction (partial or complete) in at least one symptom associated with a disorder associated with abnormal GI motility. For example, such a disorder can be GERD and a reduction in heartburn can be realized. In another embodiment, the disorder can be GERD and the subject can experience a reduction in any one or more of the symptoms of dysphagia, odynophagia, hemorrhage, water brash, esophageal erosion, esophageal obstruction and respiratory manifestations such as asthma, recurrent pneumonia, coughing, intermittent wheezing, earache, hoarseness, laryngitis and pharyngitis.

As used herein, the term “pharmaceutically acceptable excipient” includes compounds that are compatible with the other ingredients in a pharmaceutical formulation and not injurious to the subject when administered in therapeutically effective amounts.

As used herein, the term “pharmaceutically acceptable salt” includes salts that are physiologically tolerated by a subject. Such salts are typically prepared from an inorganic and/or organic acid. Examples of suitable inorganic acids include, but are not limited to, hydrochloric, hydrobromic, hydroiodic, nitric, sulfuric, and phosphoric acid. Organic acids may be aliphatic, aromatic, carboxylic, and/or sulfonic acids. Suitable organic acids include, but are not limited to, formic, acetic, propionic, succinic, camphorsulfonic, citric, fumaric, gluconic, lactic, malic, mucic, tartaric, para-toluenesulfonic, glycolic, glucuronic, maleic, furoic, glutamic, benzoic, anthranilic, salicylic, phenylacetic, mandelic, pamoic, methanesulfonic, ethanesulfonic, pantothenic, benzenesulfonic (besylate), stearic, sulfanilic, alginic, galacturonic, and the like.

As used herein, “co-administration” of first and at least one second compounds refers to the simultaneous delivery of two or more separate chemical entities, whether in vitro or in vivo (e.g., to a subject). In some embodiments, compounds that are coadministered work in conjunction with each other (e.g., to increase GI motility).

In certain embodiments, co-administration of a first amount of a compound having 5-HT₃ receptor agonist activity or a pharmaceutically acceptable salt, hydrate or solvate thereof and a second amount of at least one other compound results in an increased therapeutic effect, relative to the effect resulting from separate administration of the first amount of the compound having 5-HT₃ receptor agonist activity or a pharmaceutically acceptable salt, hydrate or solvate thereof or the second amount of at least one other compound. In some embodiments, an increased therapeutic effect is an additive effect. In some other embodiments, an increased therapeutic effect is a synergistic effect. In certain embodiments, the amounts of each of a first compound having 5-HT₃ receptor agonist activity or a pharmaceutically acceptable salt, hydrate or solvate thereof and at least one second compound, as described herein, are less than the amounts used when each compound is administered alone, thereby lessening or alleviating at least one detrimental side effect associated with single administration of either of the compounds.

Examples of a compound having 5-HT₃ receptor agonist activity include, but are not limited to, MKC-733 (i.e., monohydrochloride salt of (R)-N-1-azabicyclo[2.2.2]oct-3-yl-4,7-dihydro-7-oxothieno[3,2-b]pyridine-6-carboxamide), thieno[3,2-b]pyridine derivatives, thiazole derivatives and YM 31636, and equivalents thereof.

Examples of at least one second compound which is used in combination with a compound having 5-HT₃ receptor agonist activity include, but are not limited to, compounds having 5-HT₄ receptor agonist activity (e.g., tegaserod, cisapride, prucalopride, SL 65.0155, ATI-7505, and TD-2749); compounds having both 5-HT₄ receptor agonist activity and 5-HT₃ receptor antagonist activity (e.g., mosapride, renzapride, and E-3620); compounds having both 5-HT₄ receptor agonist activity and dopamine receptor antagonist activity (e.g., metoclopramide); compounds having both 5-HT₄ receptor agonist activity and D₂-receptor antagonist activity (e.g., itopride); compounds having only D₂-receptor antagonist activity (e.g., chlorpromazine, prochlorperazine, haloperidol, and alizapride); compounds having motilin receptor agonist activity (e.g., erythromycin, mitemcinal, and atilmotin); compounds having GABA-B receptor agonist activity (e.g., baclofen and XP-19986); compounds having corticotropin-releasing factor (CRF-1) receptor antagonist activity (e.g., CP-316311, TS-041, 876008, ONO-2333MS, AAG-561 and NBI-34041); compounds having opioid receptor antagonist activity (e.g., naltrexone, PTI-901,alvimopan, methylnaltrexone, and naloxone); compounds having alpha 2 adrenoreceptor agonist activity (e.g., clonidine, tizanidine, and guanfacine); compounds having tachykinin (NK₁, NK₂, and/or NK₃) receptor antagonist activity (e.g., nepadutant, saredutant, talnetant, and osanetant); compounds having muscarinic cholinergic receptor agonist activity (e.g. bethanechol); acetylcholinesterases inhibitors (e.g., neostigmine and carbachol); compounds having mixed serotonin and noradrenaline reuptake inhibitor activity (e.g., milnacipran, venlafaxine, desvenlafaxine, sibutramine and duloxetine); benzodiazepine-like molecules (e.g., dextofisopam, levotofisopam, diazepam, lorazepam, alprazolam and clonazepam); compounds having nitric oxide synthase (NOS) inhibitory activity (e.g., SC-81490, targinine, and 274150); cannabinoid receptor modulators (e.g., dronabinol, nabilone, rimonabant, cannabidiol, and SAB-378); compounds having reversible cholecystokinin 1 (CCK1) receptor antagonist activity (e.g., dexioxiglumide, and devazepide); compounds that activate chloride channels (e.g., lubiprostone); compounds having guanylate cyclase receptor agonist activity (e.g., MD-1100); and glucagon-like peptide (GLP) analogs (e.g. GTP-010).

In some embodiments, at least one second compound used in combination with a compound having 5HT₃ is a compound having an opioid receptor antagonist activity. In a particular embodiment, the at least one second compound is naltrexone used in combination with DDP733. In another embodiment, the at least one second compound is alvimopan used in combination with DDP733.

Exemplary combinations include a MKC-733 as a first compound and tegaserod as a second compound, wherein the combination of the two results in an increase in GI motility.

In some embodiments, a second compound has receptor modulating activity. As used herein, a “receptor” is intended to include any molecule present inside or on the surface of a cell that may affect cellular physiology when it is inhibited or stimulated by a ligand. Typically, a receptor comprises an extracellular domain with ligand-binding properties, a transmembrane domain that anchors the receptor in the cell membrane, and a cytoplasmic domain that generates a cellular signal in response to ligand binding (“signal transduction”). A receptor also includes any molecule having the characteristic structure of a receptor, but with no identifiable ligand. In addition, a receptor includes a truncated, modified, mutated receptor, or any molecule comprising part or all of the sequences of a receptor, having activity of the naturally occurring receptor.

The term “receptor modulating activity,” refers to a property of a compound which interacts with a receptor either directly or indirectly, thereby to either increase or decrease an activity of the receptor.

The term “agonist,” as used herein, refers to a compound that increases the activity of a receptor. An agonist may either directly interact (e.g., bind) with a receptor or indirectly increase its activity (e.g., to increase the availability of the endogenous neurotransmitter). An agonist refers to a compound which triggers a response by virtue of its interaction (direct or indirect) with a receptor. A “partial agonist” activates a receptor but does not cause as much of a physiological change as does a full agonist.

The term “antagonist” as used herein refers to a compound that decreases the activity of a receptor. An antagonist may either directly interact (e.g., bind) with a receptor or indirectly decrease its activity (e.g., to reduce the availability of the endogenous neurotransmitter). An antagonist also includes compounds which not only fail to activate the receptor with which they interact (directly or indirectly) but also block the receptor's activation by agonists.

Compounds for use in the methods or compositions of the invention can be formulated for oral, transdermal, sublingual, buccal, parenteral, rectal, intranasal, intrabronchial or intrapulmonary administration. In some embodiments, compositions described herein are administered orally, including but not limited to compositions including MKC-733. For oral administration, the compounds can be of the form of tablets or capsules prepared by conventional means with pharmaceutically acceptable excipients such as binding agents (e.g., polyvinylpyrrolidone or hydroxypropylmethylcellulose); fillers (e.g., lactose, microcrystalline cellulose or calcium phosphate); lubricants (e.g., magnesium stearate, talc or silica); disintegrates (e.g., sodium starch glycollate); or wetting agents (e.g., sodium lauryl sulphate). If desired, the tablets can be coated using suitable methods. Liquid preparation for oral administration can be in the form of solutions, syrups or suspensions. The liquid preparations can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

For buccal administration, the compounds for use in the methods or compositions of the invention can be in the form of tablets or lozenges formulated in a conventional manner.

For parenteral administration, the compounds for use in the methods or compositions of the invention can be formulated for injection or infusion, for example, intravenous, intramuscular or subcutaneous injection or infusion, or for administration in a bolus dose and/or infusion (e.g., continuous infusion). Suspensions, solutions or emulsions in an oily or aqueous vehicle, optionally containing other formulatory agents such as suspending, stabilizing and/or dispersing agents can be used.

For rectal administration, the compounds for use in the methods or compositions of the invention can be in the form of suppositories.

For sublingual administration, tablets can be formulated in conventional manner.

For intranasal, intrabronchial or intrapulmonary administration, conventional formulations can be employed.

Further, the compounds for use in the methods or compositions of the invention can be formulated in a sustained release preparation. For example, the compounds can be formulated with a suitable polymer or hydrophobic material which provides sustained and/or controlled release properties to the active agent compound. As such, the compounds for use the method of the invention can be administered in the form of microparticles for example, by injection or in the form of wafers or discs by implantation.

Additional dosage forms suitable for use in the methods or compositions of the invention include dosage forms as described in U.S. Patent Publication No. 20050059704, the entire content of which is incorporated herein by reference.

In some embodiments, the dosage forms of the compositions described herein include pharmaceutical tablets for oral administration as described in U.S. patent application No. 20030104053, the entire contents of which are incorporated by reference herein. The dosage forms of this invention include dosage forms in which the same drug is used both in the immediate-release and the prolonged-release portions as well as those in which one drug is formulated for immediate release and another drug, different from the first, for prolonged release.

In some embodiments, the supporting matrix in controlled-release tablets or controlled release portions of tablets is a material that swells upon contact with gastric fluid to a size that is large enough to promote retention in the stomach while the subject is in the digestive state, which is also referred to as the postprandial or “fed” mode. This is one of two modes of activity of the stomach that differ by their distinctive patterns of gastroduodenal motor activity. The “fed” mode is induced by food ingestion and begins with a rapid and profound change in the motor pattern of the upper GI tract. The change consists of a reduction in the amplitude of the contractions that the stomach undergoes and a reduction in the pyloric opening to a partially closed state. The result is a sieving process that allows liquids and small particles to pass through the partially open pylorus while indigestible particles that are larger than the pylorus are retropelled and retained in the stomach. This process causes the stomach to retain particles that are greater than about 1 cm in size for about 4 to 6 hours. The controlled-release matrix in these embodiments described herein is therefore selected as one that swells to a size large enough to be retropelled and thereby retained in the stomach, causing the prolonged release of the drug to occur in the stomach rather than in the intestines.

Compounds used in methods described herein may either be co-administered or be administered sequentially. “Co-administration,” as used herein, refers to administration of a first amount of a compound having 5-HT₃ receptor agonist activity or a pharmaceutically acceptable salt, hydrate or solvate thereof and a second amount of at least one other compound, where the first and second amounts together comprise a therapeutically effective amount to treat or prevent or manage a disorder associated with GI hypomotility or for increasing esophageal motility in a subject or for increasing the velocity of colonic propulsion in a subject or for increasing esophageal peristaltic wave amplitude in a subject in need of treatment. Co-administration encompasses administration of the first and second amounts of the compounds in an essentially simultaneous manner such as, for example, in a single pharmaceutical composition, for example, capsule or tablet having a fixed ratio of first and second amounts, or in multiple, separate capsules or tablets for each. “Sequential administration,” refers to separate administration of each compound in a sequential manner in either order. When administration involves the separate administration (e.g., sequential administration) of the first amount of the compound having 5-HT₃ receptor agonist activity and a second amount of at least one other compound, as described herein, the compounds are administered sufficiently close in time to have the desired therapeutic effect. For example, the period of time between each administration, which can result in the desired therapeutic effect, can range from minutes to hours and can be determined based on the properties of each compound such as potency, solubility, bioavailability, plasma half-life and kinetic profile. For example, a compound having 5-HT₃ receptor agonist activity and a second compound (for example, a compound having 5-HT₄ receptor agonist activity) can be administered in any order within about 24 hours of each other or within any time less than 24 hours of each other,

In some embodiments, a composition described herein is administered to a subject prior to commencement of an activity, where an increase in GI motility would be desirable. For example, in some embodiments, a composition is administered between 0 and 3 hours prior to an activity where increase in GI motility is desirable. In other embodiments, a composition is administered between 3 and 24 hours prior to an activity where increase in GI motility is desirable.

Release of the compounds can occur in the mouth, stomach, duodenum, ileum, jejunum, colon or any combinations of the above. For example, a single oral composition can be formulated such that the compound having 5-HT₃ receptor agonist activity and a second compound such as, for example, a compound having 5-HT₄ receptor agonist activity are released in the mouth, stomach, duodenum, ileum, jejunum, colon or any combinations of the above. In addition, a composition can be formulated to release a compound such as, for example, a compound having 5-HT₄ receptor agonist activity first, followed by the compound having 5-HT₃ receptor agonist activity, or vice versa. Staggered release of compounds can be accomplished in single composition using any suitable formulation technique such as those described herein. For example, a variety of coating thicknesses and/or different coating agents can provide staggered release of compounds from a single composition, and release at a desired location in the upper GI tract. In some embodiments, a single composition having two portions can be prepared. Portion 1 can include a compound having 5-HT₃ receptor agonist activity and portion 2 can include a second compound, where the combination of the two results in an increase in GI motility. A variety of formulation techniques such as gastric retention techniques, coating techniques, fast dissolving dosage forms and the use of suitable excipients and/or carriers can be utilized to achieve the desired release of compounds. Formulations described herein may include, but are not limited to, continuous, as needed, short-term, rapid-offset, controlled release, sustained release, delayed release and pulsatile release formulations.

In addition to the compounds described herein, additional therapeutic agents can be used in the methods described herein. Additional therapeutic agents can be, but are not limited to, antacids, for example, TUMS®, ROLAIDS®, H2 antagonists such as ranitidine and proton pump inhibitors such as omeprazole. Generally, the additional therapeutic agent does not diminish the effects of the therapy and/or potentiates the effects of the primary administration.

A therapeutically effective amount of a combination of a first amount of a compound having 5-HT₃ receptor agonist activity (e.g., MKC-733) and a second amount of at least one other compound as described herein (e.g., compounds having 5-HT₄ receptor agonist activity; compounds having both 5-HT₄ receptor agonist activity and 5-HT₃ receptor antagonist activity; compounds having both 5-HT₄ receptor agonist activity and dopamine receptor antagonist activity; compounds having both 5-HT₄ receptor agonist activity and D₂-receptor antagonist activity; compounds having D₂ receptor antagonist activity; compounds having GABA-B receptor agonist activity; compounds having motilin receptor agonist activity; compounds having opioid receptor antagonist activity; compounds having cholinergic receptor agonist activity; compounds having mixed serotonin and noradrenaline reuptake inhibitor (SNRI) activity; acetylcholinesterase inhibitors; benzodiazepine-like molecules; compounds having NOS inhibitory activity; compounds having corticotrophin releasing factor CRF-1 receptor antagonist activity; compounds having tachykinin receptor antagonist activity; compounds having alpha2 adrenoreceptor agonist activity; cannabinoid receptor modulators; compounds having reversible cholecystokinin (CCK) receptor antagonist activity; compounds that activate chloride channels; compounds having guanylate cyclase receptor agonist activity; glucagon-like peptide (GLP) analogs) will depend on the age, sex and weight of the patient, the current medical condition of the patient and the nature of the disorder being treated. The skilled artisan will be able to determine appropriate dosages depending on these and other factors. It is understood that because of the effect on GI motility of the combinations of compounds described herein, a therapeutically effective amount each compound in a composition is lower than the amount used when the compound is administered alone.

A suitable dose per day for each of the compounds, i.e., a first compound having 5-HT₃ receptor agonist activity (e.g., MKC-733) and a second compound as described herein (e.g., compounds having 5-HT₄ receptor agonist activity; compounds having both 5-HT₄ receptor agonist activity and 5-HT₃ receptor antagonist activity; compounds having both 5-HT₄ receptor agonist activity and dopamine receptor antagonist activity; compounds having both 5-HT₄ receptor agonist activity and D₂-receptor antagonist activity; compounds having D₂ receptor antagonist activity; compounds having GABA-B receptor agonist activity; compounds having motilin receptor agonist activity; compounds having opioid receptor antagonist activity; compounds having cholinergic receptor agonist activity; compounds having mixed serotonin and noradrenaline reuptake inhibitor (SNRI) activity; acetylcholinesterase inhibitors; benzodiazepine-like molecules; compounds having nitric oxide synthase (NOS) inhibitory activity; compounds having corticotrophin releasing factor CRF-1 receptor antagonist activity; compounds having NK receptor antagonist activity; compounds having alpha2 adrenoreceptor agonist activity; cannabinoid receptor modulators; compounds having reversible cholecystokinin (CCK) receptor antagonist activity; compounds that activate chloride channels; compounds having guanylate cyclase receptor agonist activity; glucagon-like peptide (GLP) analogs) for administration can be in the range of from about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg.

In some embodiments, dose of a compound having 5-HT₃ receptor agonist activity is between about 0.0001 mg and about 25 mg. In some embodiments, a dose of a compound having 5-HT₃ receptor agonist (e.g., MKC-733) used in compositions described herein is less than about 100 mg, or less than about 80 mg, or less than about 60 mg, or less than about 50 mg, or less than about 30 mg, or less than about 20 mg, or less than about 10 mg, or less than about 5 mg, or less than about 2 mg, or less than about 0.5 mg. Similarly, in some embodiments, a dose of a second compound as described herein is less than about 1000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg.

In some embodiments, a dose of MKC-733 used in compositions described herein is less than about 2 mg or less than about 0.5 mg.

Additional suitable doses per day for each of the compounds, i.e., a first compound having 5-HT₃ receptor agonist activity (e.g., MKC-733) and a second compound as described herein (e.g., compounds having 5-HT₄ receptor agonist activity; compounds having both 5-HT₄ receptor agonist activity and 5-HT3 receptor antagonist activity; compounds having both 5-HT₄ receptor agonist activity and dopamine receptor antagonist activity; compounds having both 5-HT₄ receptor agonist activity and D₂-receptor antagonist activity; compounds having D₂ receptor antagonist activity; compounds having GABA-B receptor agonist activity; compounds having motilin receptor agonist activity; compounds having opioid receptor antagonist activity; compounds having cholinergic receptor agonist activity; compounds having mixed serotonin and noradrenaline reuptake inhibitor (SNRI) activity; acetylcholinesterases; benzodiazepine-like molecules; compounds having NOS inhibitory activity; compounds having CRF-1 receptor antagonist activity; compounds having NK receptor antagonist activity; compounds having alpha2 adrenoreceptor agonist activity; cannabinoid receptor modulators; compounds having reversible cholecystokinin (CCK) receptor antagonist activity; compounds that activate chloride channels; compounds having guanylate cyclase receptor agonist activity; glucagon-like peptide (GLP) analogs) for administration include doses of about or greater than 1 ng, about 5 ng, about 10 ng, about 20 ng, about 30 ng, about 40 ng, about 50 ng, about 100 ng, about 200 ng, about 300 ng, about 400 ng, about 500 ng, about 1 μg, about 5 μg, about 10 μg, about 20 μg, about 30 μg, about 40 μg, about 50 μg, about 100 μg, about 200 μg, about 300 μg, about 400 μg, about 500 μg (0.5 mg), about 1 mg, about 1.25 mg, about 1.5 mg, about 2.0 mg, about 2.5 mg, about 3.0 mg, about 3.5 mg, about 4.0 mg, about 4.5 mg, about 5 mg, about 10 mg, about 20 mg, about 30 mg, about 40 mg, about 50 mg, about 100 mg, about 200 mg, about 300 mg, about 400 mg, about 500 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, or about 9,500 mg.

In some embodiments, a suitable dose of DDP733 is used in combination with a suitable dose of an opioid receptor antagonist, e.g., naltrexone. In another embodiment, a suitable dose of DDP733 is used in combination with a suitable dose of an opioid receptor antagonist, e.g., alvimopan.

Also encompassed by this invention are kits for treating, preventing and/or managing a disorder associated with GI hypomotility. Such a kit includes, for example, a compound having 5-HT₃ receptor agonist activity and at least one other compound chosen from: a compound having 5-HT₄ receptor agonist activity; a compound having both 5-HT₄ receptor agonist activity and 5-HT₃ receptor antagonist activity; a compound having both 5-HT₄ receptor agonist activity and dopamine receptor antagonist activity; a compound having both 5-HT₄ receptor agonist activity and D₂-receptor antagonist activity; a compound having D₂ receptor antagonist activity; a compound having (GABA)-B receptor agonist activity; a compound having motilin receptor agonist activity; a compound having opioid receptor antagonist activity; a compound having cholinergic receptor agonist activity; a compound having mixed serotonin and noradrenaline reuptake inhibitor (SNRI) activity; acetylcholinesterase inhibitors; benzodiazepine-like molecules; a compound having NOS inhibitory activity; a compound having corticotrophin releasing factor CRF1 receptor agonist activity; a compound having tachykinin receptor agonist activity; a compound having alpha2 receptor agonist activity; a cannabinoid modulator; compounds having reversible cholecystokinin (CCK) receptor antagonist activity; compounds that activate chloride channels; compounds having guanylate cyclase receptor agonist activity; and glucagon-like peptide (GLP) analogs). Kits might further include a device, for example, for administering the compounds described herein. Additionally, kits may include instructions for administration of one or more compounds in the compositions and/or promotional materials such as, for example, marketing materials and/or any documents promoting the use of the compounds in the compositions.

Also featured by this invention are kits for treating, preventing and/or managing a disorder associated with GI hypomotility, which include, for example, a compound having 5-HT₃ receptor agonist activity and instructions and/or promotional materials for using the compound in combination with at least one other compound chosen from: a compound having 5-HT₄ receptor agonist activity; a compound having both 5-HT₄ receptor agonist activity and 5-HT₃ receptor antagonist activity; a compound having both 5-HT₄ receptor agonist activity and dopamine receptor antagonist activity; a compound having both 5-HT₄ receptor agonist activity and D₂-receptor antagonist activity; a compound having D₂ receptor antagonist activity; a compound having (GABA)-B receptor agonist activity; a compound having motilin receptor agonist activity; a compound having opioid receptor antagonist activity; a compound having cholinergic receptor agonist activity; a compound having mixed serotonin and noradrenaline reuptake inhibitor (SNRI) activity; an acetylcholinesterase inhibitor; benzodiazepine-like molecules; a compound having NOS inhibitory activity; a compound having corticotrophin releasing factor CRF1 receptor agonist activity; a compound having tachykinin receptor agonist activity; a compound having alpha2 receptor agonist activity; a cannabinoid modulator; a compound having reversible cholecystokinin (CCK) receptor antagonist activity; a compound that activate chloride channels; a compounds having guanylate cyclase receptor agonist activity; and a glucagon-like peptide (GLP) analog.

In a particular embodiment, a kit for treating, preventing or managing a gastrointestinal hypomotility disorder featured herein includes a compound having 5-HT₃ receptor agonist activity (e.g., DDP733) and instructions and/or promotional materials for using the compound in combination with a compound having opioid receptor antagonist activity (e.g., naltrexone or alvimopan).

In another embodiment, a kit includes at least one compound selected from: a compound having 5-HT₄ receptor agonist activity; a compound having both 5-HT₄ receptor agonist activity and 5-HT₃ receptor antagonist activity; a compound having both 5-HT₄ receptor agonist activity and dopamine receptor antagonist activity; a compound having both 5-HT₄ receptor agonist activity and D₂-receptor antagonist activity; a compound having D₂ receptor antagonist activity; a compound having (GABA)-B receptor agonist activity; a compound having motilin receptor agonist activity; a compound having opioid receptor antagonist activity; a compound having cholinergic receptor agonist activity; a compound having mixed serotonin and noradrenaline reuptake inhibitor (SNRI) activity; an acetylcholinesterase inhibitor; a benzodiazepine-like molecule; a compound having NOS inhibitory activity; a compound having corticotrophin releasing factor CRF1 receptor agonist activity; a compound having tachykinin receptor agonist activity; a compound having alpha2 receptor agonist activity; a cannabinoid modulator; a compound having reversible cholecystokinin (CCK) receptor antagonist activity; a compound that activate chloride channels; a compound having guanylate cyclase receptor agonist activity; and a glucagon-like peptide (GLP) analog with instructions and/or promotional materials for using the compound in combination with a compound having 5-HT₃ receptor agonist activity.

In one embodiment, a kit featured herein includes at least one compound having opioid receptor antagonist (e.g., naltrexone or alvimopan) with instructions and/or promotional materials for using the compound in combination a compound having 5-HT₃ receptor agonist activity (e.g., DDP733).

In one embodiment, kits featured herein include instructions and/or promotional materials for administration with an additional therapeutic agent based upon the functional relationship between the agents. For example, a compound having 5-HT₃ receptor agonist activity (e.g., DDP733) may be packaged with an instructional insert which details the administration of the compound with a second compound (e.g., a compound having opioid antagonist activity) such that they work synergistically. In other examples, a compound having 5-HT₃ receptor agonist activity (e.g., DDP733) may be packaged with an instructional insert and/or promotional materials which details the administration of the compound with a second compound such that they work additively. In still other examples, a compound having 5-HT₃ receptor agonist activity (e.g., DDP733) may be packaged with an instructional insert which details the administration of the compound with a second compound and further in combination with a carrier or other therapeutic agent such that their activities do not interfere with each other. It is understood that in practicing the method or using a kit of the present invention that administration encompasses administration by different individuals (e.g., the subject, physicians or other medical professionals) administering the same or different compounds.

It is understood that compounds having the various activities described herein can be identified, for example, by screening libraries or collections of molecules using suitable methods. Another source for the compounds of interest are combinatorial libraries which can comprise many structurally distinct molecular species. Combinatorial libraries can be used to identify lead compounds or to optimize a previously identified lead. Such libraries can be manufactured by well-known methods of combinatorial chemistry and screened by suitable methods.

This invention is further illustrated by the following examples which should not be construed as limiting. The contents of all references, patents and published patent applications cited throughout this application are incorporated herein by reference.

### EXAMPLES

### Example I

### Combination of a 5HT₃ Receptor Agonist and an Opioid Receptor Antagonist Reverse a Morphine-induced Decrease in Pellet Propulsion in a Guinea Pig Ex Vivo Colon Preparation

Materials and Methods

I. Animals

Hartley guinea pigs of either sex (Charles River Laboratories, Wilmington, Mass.) weighing approximately 300 grams were used in all experiments described herein. Segments of distal colon were taken from guinea pigs following euthanasia by isoflurane anesthesia and exsanguination. Colon segments were immediately placed on ice-cold physiological saline solution (PSS, see below for composition).

II. Solutions, Drugs, and Reagents

PSS was made daily and consisted of (in mM) 119 NaCl, 4.7 KCl, 24 NaHCO₃, 1.2 KH₂PO₄, 2.5 CaCl₂, 1.2 MgSO₄, 11 glucose, and aerated with 95% O₂-5% CO₂ to obtain pH 7.4. Morphine (Sigma Chemical Company, St. Louis, Mo.), Naltrexone (Sigma Chemical Company, St. Louis, Mo.) or DDP733 (Dynogen Pharmaceuticals, Inc., Waltham, Mass.) were dissolved as stock solutions in distilled water. Working concentrations of drugs were prepared by dissolving the stock into PSS used in each experiment.

III. Statistics and Data Analysis

Data are reported as means±SEM. Data were analyzed by t-test.

IV. Fecal Pellet Propulsion Velocity Assay

Segments of guinea-pig distal colon (5-8 cms long) were pinned out in an organ bath with pins attached to small pieces of mesentery adhering to the wall of the gut at intervals of approximately 1.5-2 cms. Colonic segments were superfused with oxygenated PSS warmed to about 37° C. An equilibration period was allowed, during which time the gut developed spontaneous tone and emptied its luminal contents. Following the equilibration period, a PE-50 catheter was inserted approximately 1 to 2 cms into the lumen via the anal end of the colon. Oxygenated PSS was perfused intraluminally at a rate of about 0.15 ml/min for a period of 30 minutes. At this point, a dried fecal pellet that had been coated with a synthetic polymer to maintain its structural integrity, was placed into the oral end of the colon, and the intraluminal catheter was advanced to the distal edge of the fecal pellet. The amount of time for the fecal pellet to move about 2 to 3 cms in the aboral direction was measured within the center of the colon segment using a custom computer program and digital video camera. A total of three motility trials were conducted in each experiment under baseline conditions, with a five-minute recovery period between each trial. Motility rates from these first three trials were averaged together to give a measure of baseline motility. After the third baseline trial, a 30-minute inter-trial equilibration period was allowed

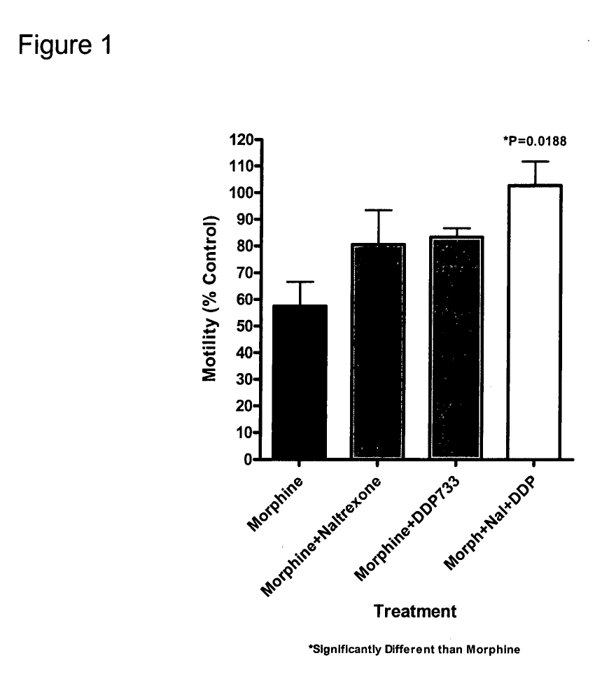

PSS containing either naltrexone (an opioid antagonist) or DDP733 (a 5-HT₃ agonist) was infused intraluminally prior to the end of the 30 minute inter-trial equilibration period (e.g., 2 minutes prior to start of motility trials for DDP733 and 15 minutes prior to start of motility trials for naltrexone). At the end of the equilibration period, a fecal pellet was inserted into the oral end of the colon, and motility rate was assessed as before. A total of three motility trials were performed, with a 5-minute recovery period between each trial. Intraluminal infusion of naltrexone or DDP733 was resumed after each trial. Each colon was exposed to only a single concentration of naltrexone or DDP733. The average motility rate in the presence of naltrexone or DDP733 was expressed as a percentage of the average baseline motility rate in each specimen (% Control), as shown in FIG. 1 and Table II.

Morphine (0.01 to 10 μM) was added to the superfusate (bath solution) during the inter-trial equilibration period, e.g., 15 minutes prior to the start of motility trials. Morphine remained in the bath solution for the remainder of the experiment. At the end of the equilibration period, a fecal pellet was inserted into the oral end of the colon, and motility rate was assessed as before. A total of three motility trials were performed, with a 5-minute recovery period between each trial. Each colon was exposed to only a single concentration of morphine. The average motility rate in the presence of morphine was expressed as a percentage of the average baseline motility rate in each preparation (% Control), as shown in FIG. 1 and Table II.

Morphine and naltrexone (0.01 μM and 1 μM, respectively) were added to the superfusate (bath solution) during the inter-trial equilibration period, about 15 minutes prior to the start of motility trials. Naltrexone was also infused intraluminally at this point. Morphine and naltrexone remained in the bath solution for the remainder of the experiment. At the end of the equilibration period, a fecal pellet was inserted into the oral end of the colon, and motility rate was assessed as before. A total of three motility trials were performed, with a 5-minute recovery period between each trial. The average motility rate in the presence of morphine and naltrexone was expressed as a percentage of the average baseline motility rate in each preparation (% Control), as shown in FIG. 1 and Table II.

Morphine (0.01 μM) was added to the superfusate (bath solution) during the inter-trial equilibration period, 15 min prior to the start of motility trials. At this point, DDP733 (1 μM or 10 μM) was infused intraluminally. Morphine and remained in the bath solution for the remainder of the experiment, and DDP733 was infused intraluminally for the remainder of the experiment. At the end of the equilibration period, a fecal pellet was inserted into the oral end of the colon, and motility rate was assessed as before. A total of three motility trials were performed, with a 5-minute recovery period between each trial. The average motility rate in the presence of morphine and DDP733 was expressed as a percentage of the average baseline motility rate in each preparation (% Control), as shown in FIG. 1 and Table II.

Table II below summarizes the data from one such experiment with the motility represented as a percentage (%) of control.

TABLE II

| | Morphine | Morphine | Morphine | Morphine |
|---|---|---|---|---|
| Compound(s) | 0.01 μM | 0.10 μM | 1.0 μM | 10 μM |
| Motility | 31 | 72 | 104 | 95 |
| % control | 63 | 57 | 0.0 | 0 |
| | 73 | 73 | 20 | 82 |
| | 63 | 78 | 34 | 0 |

| | | | | Morphine 0.01 μM, | Morphine 0.01 μM, |
|---|---|---|---|---|---|
| | Morphine 0.01 μM | Morphine 0.01 μM | Morphine 0.01 μM | naltrexone 1 μM, | naltrexone 1 μM, |
| Compound(s) | and naltrexone 1 μM | and DDP733 1 μM | and DDP733 10 μM | and DDP733 1 μM | and DDP733 10 μM |
| Motility | 54 | 80 | 98 | 86 | 108 |
| % control | 78 | 80 | 113 | 105 | 63 |
| | 116 | 90 | | 117 | 88 |
| | 74 | | | | |

As elicited by Table II, morphine leads to a decrease in motility, an effect which is diminished in the presence of DDP733 and further in presence of both DDP733 and naltrexone.

These results suggest that a combination of a 5-HT₃ receptor agonist and an opioid receptor antagonist will be effective in treating gastrointestinal hypomotility and associated disorders including but not limited to, for example, gastroesophageal reflux disease (GERD), nocturnal gastroesophageal reflux disease (n-GERD), dyspepsia, constipation including slow transit constipation, normal transit constipation, acute constipation, chronic idiopathic constipation, opioid-induced constipation, constipation associated with irritable bowel syndrome and constipation due to increased tone of the large intestine, ileus and post-operative ileus, narcotic bowel syndrome, gastroparesis including diabetic gastroparesis and intestinal pseudo-obstruction gastroparesis, Hirschsprung's disease, decreased peristalsis of the esophagus and/or stomach and/or the small and/or the large intestine, esophagitis, non-ulcer dyspepsia, pseudo-obstruction of the bowels and/or the colon, impaired colonic transit, epigastric pain, recurrent nausea and vomiting, anorexia nervosa, dyskinesias of the biliary system, tachygastria and problems of gastric emptying due to tachygastria, intestinal spasms and cramps, irritable bowel syndrome with diarrhea, peptic ulcer diseases, inflammatory diseases of the stomach and bowel including gastritis, duodenitis, inflammatory bowel disease, intestinal and post-operative intestinal atony, and drug-induced delayed transit.

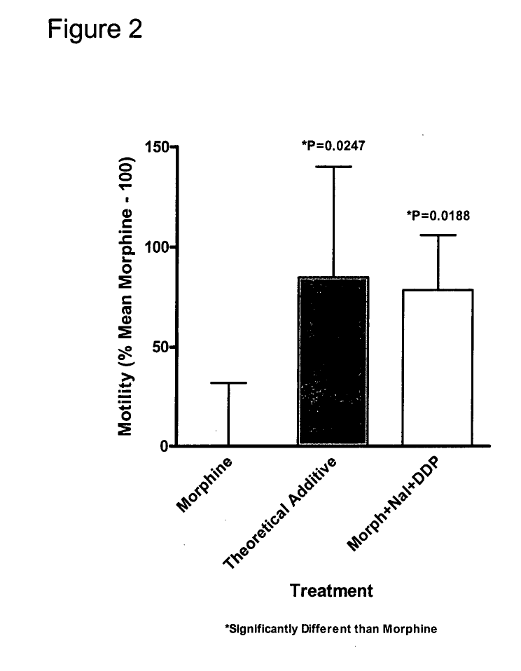

In a further step, statistical analysis was performed on the data from an exemplary experiment measuring the effect of DDP733 and/or naltrexone on morphine-induced motility to determine whether the effect of DDP733 and/or naltrexone was additive. FIG. 2 demonstrates that the motility in the presence of Morphine+Naltrexone+DDP733 was significantly higher relative to Morphine alone. The actual data was further comparable to a theoretical additive value.

The statistical analysis demonstrates that the effect of a combination of a 5-HT₃ receptor agonist (e.g., DDP733) and an opioid receptor antagonist (e.g., naltrexone) on morphine-induced hypomotility is statistically significant and additive.

### Example 2

### Combination of a 5-HT₃ Receptor Agonist and a Second Compound Increases Pellet Propulsion in Isolated Colons of Guinea Pigs

The distal colon of male guinea pigs (weight 150-200 g) is incubated at 37° C. for 30 mins in Krebs-bicarbonate medium to allow spontaneous evacuation of fecal pellets. The composition of the medium used is as follows: 118 NaCl mM; 4.8 mM KCl; 1.2 mM KH₂PO₄; 1.2 mM MgSO₄; 2.5 mMCaCl₂; 25 mM NaHCO₃, and 11 mM glucose. The colon is then cut into segments of equal length and each segment is secured with pins placed at intervals through the attached mesentery.

Artificial clay pellets, which mimic natural colonic pellets in size and shape (approximately 10 mm long×4 mm wide), are used to measure the velocity of propulsion. Control (i.e., basal) velocity is measured by inserting a pellet into the orad end of a colonic segment and allowing it to exit spontaneously through the caudad opening of the segment. The velocity is calculated from the time taken by a pellet to traverse a marked segment. At 5-min intervals, a second and then a third pellet are inserted into the orad end, and the measurement of velocity is repeated. Control velocity is taken as the mean velocity of propulsion of pellets. The segments are then allowed to equilibrate again for 30 min in fresh Krebs-bicarbonate solution. After the equilibration period, various compounds, as described herein, are applied and the velocity of propulsion of pellets is measured. The effect of a first compound having 5-HT₃ receptor agonist activity alone and in combination with at least one second compound chosen from: compounds having 5-HT₄ receptor agonist activity; compounds having both 5-HT₄ receptor agonist activity and 5-HT₃ receptor antagonist activity; compounds having both 5-HT₄ receptor agonist activity and dopamine receptor antagonist activity; compounds having both 5-HT₄ receptor agonist activity and D₂-receptor antagonist activity; compounds having D₂ receptor antagonist activity; compounds having GABA-B receptor agonist activity; compounds having motilin receptor agonist activity; compounds having opioid receptor antagonist activity; compounds having cholinergic receptor agonist activity; compounds having mixed serotonin and noradrenaline reuptake inhibitor (SNRI) activity; acetylcholinesterase inhibitors; benzodiazepine-like molecules; compounds having NOS inhibitory activity; compounds having corticotrophin CRF-1 receptor antagonist activity; compounds having tachykinin receptor antagonist activity; compounds having alpha2 adrenoreceptor agonist activity; cannabinoid receptor modulators; compounds having reversible cholecystokinin (CCK) receptor antagonist activity; compounds that activate chloride channels; compounds having guanylate cyclase receptor agonist activity; glucagon-like peptide (GLP) analogs), is determined.

Test compounds may be perfused intraluminally and/or added to the bathing medium. In all studies the response to a given concentration of agonist or antagonist is measured in a single segment.

Results are expressed as percent of control (i.e., basal) velocity in millimeters per second. The concentration causing 50% of maximal response (EC50) is calculated from the fit of concentration-response curves. Values are means±SE of at least 10 colonic segments. Statistical significance can be evaluated using, for example, Student's t-test for paired or unpaired data.

In one experiment, a combination of a compound having 5-HT₃ agonist activity (i.e., MKC-733) and a compound having 5-HT₄ receptor agonist activity (i.e., tegaserod) agonist results in an increase in the velocity of colonic propulsion compared with that seen after application of either agent given alone at the same concentration. Tegaserod, derivatives thereof and other similar compounds having 5-HT₄ receptor agonist activity are described in U.S. Pat. No. 5,510,353, the entire content of which is incorporated herein by reference.

The specification is most thoroughly understood in light of the teachings of the references cited within the specification which are hereby incorporated by reference. The embodiments within the specification provide an illustration of embodiments in this disclosure and should not be construed to limit its scope. The skilled artisan readily recognizes that many other embodiments are encompassed by this invention. All publications and patents cited and sequences identified by accession or database reference numbers in this disclosure are incorporated by reference in their entirety. To the extent that the material incorporated by reference contradicts or is inconsistent with the present specification, the present specification will supercede any such material. The citation of any references herein is not an admission that such references are prior art to the present disclosure.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating, preventing or managing GI hypomotility in a subject comprising administering to the subject:
(a) a first compound having 5-HT₃ receptor agonist activity or a pharmaceutically acceptable salt, hydrate or solvate thereof; and
(b) at least one second compound chosen from: a compound having 5-HT₄ receptor agonist activity; a compound having both 5-HT₄ receptor agonist activity and 5-HT₃ receptor antagonist activity; a compound having both 5-HT₄ receptor agonist activity and dopamine receptor antagonist activity; a compound having both 5-HT₄ receptor agonist activity and D₂-receptor antagonist activity; a compound having D₂ receptor antagonist activity; a compound having (GABA)-B receptor agonist activity; a compound having motilin receptor agonist activity; a compound having opioid receptor antagonist activity; a compound having cholinergic receptor agonist activity; a compound having corticotrophin releasing factor CRF1 receptor agonist activity; a compound having tachykinin receptor agonist activity; a compound having alpha2 receptor agonist activity, an acetylcholinesterase inhibitor; a compound having mixed serotonin and noradrenaline reuptake inhibitor activity; a benzodiazepine-like molecule; a compound having nitric oxide synthase (NOS) inhibitory activity; a cannabinoid modulator; a compound having reversible cholecystokinin (CCK) receptor antagonist activity; a chloride channel activator; a compound having guanylate cyclase receptor agonist activity; and glucagon-like peptide (GLP) analogs,
wherein the combination of the first and the at least one second compound results in an increase in GI motility relative to the GI motility in presence of either the first or the at least one second compound alone.

2. The method according to claim 1, wherein increasing GI motility comprises increasing esophageal motility in the subject.

3. The method according to claim 1, wherein increasing GI motility comprises increasing colonic propulsion in the subject.

4. The method according to claim 1, wherein increasing GI motility comprises increasing esophageal peristaltic wave amplitude in the subject.

5. The method according to any of claims 1-4, wherein the first compound
having a 5-HT₃ receptor agonist activity is chosen from MKC-733, a thiazole derivative, a thieno[3,2-b]pyridine derivative, and YM 31636.

6. The method according to claims 1-4, wherein amounts of the first compound and the at least one second compound administered are lower than amounts used when only the first compound or the at least one second compound are administered.

7. The method according to any of claims 1-4, wherein the disorder is chosen from gastroesophageal reflux disease (GERD), nocturnal gastroesophageal reflux disease (n-GERD), dyspepsia, constipation, slow transit constipation, normal transit constipation, acute constipation, chronic idiopathic constipation, constipation associated with irritable bowel syndrome, constipation due to increased tone of the large intestine, opioid-induced constipation, ileus, post-operative ileus, narcotic bowel syndrome, gastroparesis, diabetic gastroparesis, intestinal pseudo-obstruction gastroparesis, Hirschsprung's disease, decreased peristalsis of the esophagus, decreased peristalsis of the stomach, decreased peristalsis of the small intestine, decreased peristalsis of the large intestine, esophagitis, non-ulcer dyspepsia, pseudo-obstruction of the bowels, pseudo-obstruction of the colon, impaired colonic transit, epigastric pain, postoperative gut atony, recurrent nausea, recurrent vomiting, anorexia nervosa, dyskinesias of the biliary system, tachygastria, problems of gastric emptying due to tachygastria, intestinal spasms, intestinal cramps, irritable bowel syndrome with diarrhea, peptic ulcer diseases, inflammatory diseases of the stomach, inflammatory bowel disease, gastritis, duodenitis, intestinal atony, post-operative intestinal atony, and drug-induced delayed transit.

8. The method according to any of claims 1-4, wherein the first and the at least one second compound are co-administered to the subject.

9. The method according to any of claims 1-4, wherein the first and the at least one second compound are administered sequentially to the subject.

10. The method according to any of claims 1-4, wherein the first and the at least one second compound are administered orally, nasally, rectally, intravaginally, parenterally, buccally, sublingually or topically.

11. The method according to any of claims 1-4, wherein the first and the at least one second compound are formulated using one or more pharmaceutically acceptable excipients chosen from starch, sugar, cellulose, diluent, granulating agent, lubricant, binder, disintegrating agent, wetting agent, emulsifier, coloring agent, release agent, coating agent, sweetening agent, flavoring agent, perfuming agent, preservative, antioxidant, plasticizer, gelling agent, thickener, hardener, setting agent, suspending agent, surfactant, humectant, carrier, stabilizer, or a combination thereof.

12. The method according to any of claims 1-4, wherein the first and the at least one second compound are each administered from one to five times per day.

13. The method according to any of claims 1-4, wherein the first and the at least one second compound are each administered one time per day.

14. The method according to any of claims 1-4, wherein the subject is a mammal.

15. The method according to any of claims 1-4, wherein the subject is a human.

16. The method according to any of claims 1-4, wherein the first compound is a thieno[3,2-b]pyridine derivative and the at least one second compound is a compound having 5-HT₄ receptor agonist activity.

17. The method according to any of claims 1-4, wherein the first compound is MKC-733 and the at least one second compound is tegaserod.

18. The method according to claim 1, wherein at least one detrimental side effect associated with administration of the first compound or administration of the at least one second compound alone is lessened by concurrent administration of the first and the second compounds.

19. A composition for treating, preventing or managing GI hypomotility comprising a first compound having 5-HT₃ receptor agonist activity and at least one second compound having 5-HT₄ receptor agonist activity.

20. The composition of claim 19, wherein the first compound is a thieno[3,2 b]pyridine derivative.

21. The composition of claim 20, wherein the thieno[3,2 b]pyridine derivative is MKC-733.

22. The composition of claim 19, wherein the at least one second compound is tegaserod.

23. A composition for treating, preventing or managing a disorder associated with GI hypomotility comprising therapeutically effective amounts of MKC-733 and tegaserod.

24. A composition for treating, preventing or managing a disorder associated with GI hypomotility comprising therapeutically effective amounts of MKC-733 and naltrexone, naloxone, naloxone methiodide, quaternary naltrexone (Q-naltrexone), cyprodime, nor-binaltorphimine, HS-378, naltrindole or alvimopan.

25. A composition for treating, preventing or managing a disorder associated with GI hypomotility comprising a therapeutically effective amount of DDP733 and a therapeutically effective amount of naltrexone or alvimopan.

26. A composition for treating, preventing or managing a disorder associated with GI hypomotility comprising therapeutically effective amounts of DDP733 and naltrexone.

27. A composition for treating, preventing or managing a disorder associated with GI hypomotility comprising therapeutically effective amounts of DDP733 and alvimopan.

28. The composition of any one of claims 24-27, wherein the disorder is selected from the group consisting of constipation including slow transit constipation, normal transit constipation, acute constipation, chronic idiopathic constipation, opioid-induced constipation, constipation associated with irritable bowel syndrome, ileus, post-operative ileus, and narcotic bowel syndrome.
